(19) United States
(12) Patent Application Publication
(10) Pub. No.: US 2022/0016011 A1
(43) Pub. Date: Jan. 20, 2022

(54) COMPOSITION OF ACTIVE INGREDIENTS, FOR IMPROVING DEPOSITION OF ANTIOXIDANTS

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE)

(73) Assignee: Henkel AG & Co. KGaA, Duesseldorf (DE)

(21) Appl. No.: 17/290,213

(22) PCT Filed: Oct. 31, 2019
(86) PCT No.: PCT/EP2019/079770

§ 371 Date: Apr. 29, 2021

(30) Foreign Application Priority Data
Oct. 31, 2018 (DE) 10 2018 127 280.4

(51) Int. Cl. A61K 8/58; A61K 8/89; A61Q 5/02; A61Q 5/06

(52) U.S. Cl. CPC A61K 8/585; A61Q 5/06; A61Q 5/02; A61K 8/89

(57) ABSTRACT

The present disclosure relates to an active ingredient composition for the care of human hair. In particular, the present disclosure relates to a cosmetic composition for treating a keratinous material comprising a) at least one organic silicon compound and b) at least one antioxidant, the cosmetic composition being particularly suitable for caring for damaged hair.

### CROSS-REFERENCE TO RELATED APPLICATION

This application is a U.S. National-Stage entry under 35 U.S.C. § 371 based on International Application No. PCT/EP2019/079770, filed Oct. 31, 2019, which was published under PCT Article 21(2) and which claims priority to German Application No. 10 2018 127 280.4, filed Oct. 31, 2018, which are all hereby incorporated in their entirety by reference.

### TECHNICAL FIELD

The present disclosure relates to cosmetic compositions for treating a keratinous material, the composition comprising as a first component an organic silicon compound and as a second component an antioxidant, and to the use of the cosmetic composition.

### BACKGROUND

The external exposure of hair to chemicals from a variety of different sources poses challenges for the development of cosmetic care products. Air and water impurities have a detrimental effect on skin and hair. Major air pollutants include polycyclic aromatic hydrocarbons, volatile organic compounds, nitrogen oxides (NOx), particulate matter, and cigarette smoke. The effect of various air pollutants can be enhanced in the presence of other air pollutants and when exposed to UV radiation.

It is known that the toxicity of gaseous pollutants in the air, such as sulfur dioxide, ozone, and nitrogen oxides, is related to their initiator activity for free radicals, which cause damage to living organisms. Free radicals are metabolic products that also occur naturally in the body. In large quantities, free radicals can promote irritation and inflammation and accelerate the process of aging. In this case, the term “oxidative damage” is used. Free radicals can also cause hair damage, which is visible, for example, as a reduction in shine as well as grip and/or fading of hair color.

Furthermore, often changing consumer demands for a certain hair texture are associated with recurring chemical exposure of the hair. For example, hair coloring stresses the hair, due to which a special, intensive care may be necessary.

In the prior art, organosilicon compounds from the group of silanes comprising at least one hydroxy group and/or hydrolysable group are described. Due to the presence of the hydroxy groups and/or hydrolysable groups, the silanes are reactive substances that hydrolyze or oligomerize or polymerize in the presence of water. The oligomerization or polymerization of the silanes initiated by the presence of the water, when applied to a keratinous material, ultimately leads to the formation of a film that can exert a protective effect.

### BRIEF SUMMARY

Disclosed is cosmetic composition for treating a keratinous material comprising: at least one organic silicon compound and at least one antioxidant.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The task underlying the present disclosure is to provide a product with an improved care and/or protection effect. In particular, the present disclosure was based on the task of providing a cosmetic agent which, after a hair treatment, enables a caring after-treatment which provides special care to stressed hair.

This task is solved by a cosmetic agent for the treatment of a keratinous material, comprising

a) at least one organic silicon compound and
b) at least one antioxidant.

By a keratinous material is meant hair, the skin, the nails (such as fingernails and/or toenails). Wool, furs, and feathers also fall under the definition of keratinous material.

In an exemplary embodiment, a keratinous material is understood to mean human hair, human skin, and human nails, in particular fingernails and toenails. In particular embodiments, keratinous material is understood to mean human hair, in particular head and/or beard hair.

As a first ingredient essential to the present disclosure, the cosmetic composition for treating a keratinous material contains at least one organic silicon compound. Suitable organic silicon compounds are selected from silanes having one, two or three silicon atoms, wherein the organic silicon compound includes one or more hydroxyl groups and/or hydrolysable groups per molecule.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds are compounds containing one to three silicon atoms. Organic silicon compounds in an exemplary embodiment contain one or two silicon atoms.

According to IUPACrules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The agent for treating a keratinous material contains at least one organic silicon compound in an exemplary embodiment selected from silanes having one, two or three silicon atoms, wherein the organic silicon compound includes one or more hydroxyl groups or hydrolysable groups per molecule.

In a most suitable embodiment, the agent for treating a keratinous material includes at least one organic silicon compound selected from silanes having one, two or three silicon atoms, wherein the organic silicon compound further includes one or more basic groups and one or more hydroxyl groups or hydrolysable groups per molecule.

This basic group can be, for example, an amino group, an alkylamino group or a dialkylamino group, which is in an exemplary embodiment connected to a silicon atom via a linker. The basic group is in an exemplary embodiment an amino group, a C₁-C₆ alkylamino group or a di(C₁-C₆)alkylamino group.

The hydrolysable group(s) is (are) in an exemplary embodiment a C₁-C₆ alkoxy group, especially an ethoxy group or a methoxy group. It is suitable when the hydrolysable group is directly bonded to the silicon atom. For example, if the hydrolysable group is an ethoxy group, the organic silicon compound in an exemplary embodiment contains a structural unit R′R″R′″Si—O—CH₂—CH₃. The residues R′, R″ and R′″ represent the three remaining free valences of the silicon atom.

Particularly good results were obtained when the agent for treating a keratinous material contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another very particularly suitable embodiment, the agent for treating a keratinous material includes at least one organic silicon compound of formula (I) and/or (II),

R₁R₂N-L-Si(OR₃)ₐ(R₄)_{b} (I),
where
R₁, R₂ both represent a hydrogen atom,
L represents a linear, two-band C₁-C₆-alkylene group, in an exemplary embodiment a propylene group (—CH₂—CH₂—CH₂—) or an ethylene group (—CH₂—CH₂—),
R₃, R₄ independently represent a methyl group or an ethyl group,
a stands for the number 3 and
b stands for the number 0.

(R₅O)_{c}(R₆)_{d}Si-(A)_{c}-[NR₇-(A′)]_{f}-[O-(A″)]_{g}-[NR₈-(A′″)]ₕ-Si(R₆′)_{d′}(OR₅′)_{c′}, (II),
where
R₅, R₅′, R₅″ independently represent a hydrogen atom or a C₁-C₆ alkyl group,
R₆, R₆′ and R₆″ independently represent a C₁-C₆ alkyl group,
A, A′, A″, A′″ and A″″ independently represent a linear or branched C₁-C₂₀ divalent alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d}″(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
provided that at least one of e, f, g, and h is different from 0.

The substituents R₁, R₂, R₃, R₄, R₅, R₅′, R₅″, R₆, R₆′, R₆″, R₇, R₈, L, A′, A″″ and A″″ in the compounds of formula (I) and (II) are explained below as examples:

Examples of a C₁-C₆ alkyl group are the groups methyl, ethyl, propyl, isopropyl, n-butyl, s-butyl, and t-butyl, n-pentyl and n-hexyl. Propyl, ethyl, and methyl are suitable alkyl radicals. Examples of a C₂-C₆ alkenyl group are vinyl, allyl, but-2-enyl, but-3-enyl and isobutenyl, suitable C₂-C₆ alkenyl radicals are vinyl and allyl. Suitable examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly suitable. Examples of an amino C₁-C₆ alkyl group are the aminomethyl group, the 2-aminoethyl group, the 3-aminopropyl group. The 2-aminoethyl group is particularly suitable. Examples of a linear two-band C₁-C₂₀ alkylene group include the methylene group (—CH₂), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly suitable. From a chain length of 3 C atoms, divalent alkylene groups can also be branched. Examples of branched two-band C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L- which stands for a linear or branched, two-band C₁-C₂₀ alkylene group.

In an exemplary embodiment, -L- represents a linear, divalent C₁-C₂₀ alkylene group. Further in an exemplary embodiment, -L- represents a linear divalent C₁-C₆ alkylene group. Particularly suitable -L stands for a methylene group (CH 2-), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—)

The organic silicon compounds of formula (I)

R₁R₂N-L-Si(OR₃)ₐ(R₄)_{b} (I),

carry the silicon-containing grouping —Si(OR₃)ₐ(R₄)_{b} at one end.

In the terminal structural unit —Si(OR₃)ₐ(R₄)_{b}, R₃ is hydrogen or C₁-C₆ alkyl group, and R₄ is C₁-C₆ alkyl group. R3 and R₃ independently of each other represent a methyl group or an ethyl group.

Here a stands for an integer from about 1 to about 3, and b stands for the integer 3−a. If a stands for the number 3, then b is equal to 0. If a stands for the number 2, then b is equal to 1. If a stands for the number 1, then b is equal to 2.

The best protection against the negative effects of water and/or air pollution (“anti-pollution” effect) and the best care of stressed hair could be obtained if the agent for treating a keratinous material contains at least one organic silicon compound of formula (I) in which the radicals R₃, R₄ independently represent a methyl group or an ethyl group.

Particularly well-suited organic silicon compounds of formula (I) are
(3-Aminopropyl)triethoxysilan

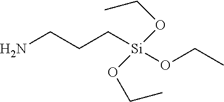

(3-Aminopropyl)trimethoxysilane

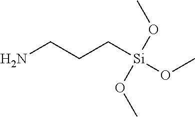

1-(3-Aminopropyl)silantriol

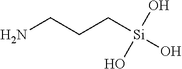

(2-Aminoethyl)triethoxysilan

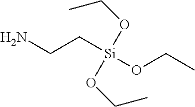

(2-Aminoethyl)trimethoxysilane

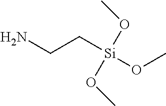

1-(2-Aminoethyl)silantriol

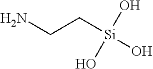

(3-Dimethylaminopropyl)triethoxysilan

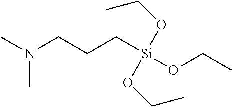 [SMILES: CCO[Si](CCCN(C)C)(OCC)OCC]

(3-Dimethylaminopropyl)trimethoxysilane

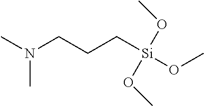

1-(3-Dimethylaminopropyl)silantriol

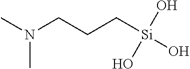

(2-Dimethylaminoethyl)triethoxysilan.

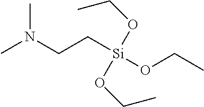

(2-Dimethylaminoethyl)trimethoxysilane and/or

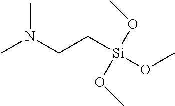

1-(2-Dimethylaminoethyl)silantriol

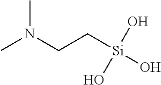

The organic silicon compound of formula (I) is commercially available. (3-aminopropyl)trimethoxysilane, for example, can be purchased from Sigma-Aldrich. (3-Aminopropyl)triethoxysilane is also commercially available from Sigma-Aldrich.

In another embodiment, the composition for treating a keratinous material includes at least one organic silicon compound of formula (II)

(R₅O)_{c}(R₆)_{d}Si-(A)_{c}-[NR₇-(A′)]_{f}-[O-(A″)]_{g}-[NR₈-(A′″)]ₕ-Si(R₆′)_{d′}(OR₅′)_{c′} (II).

The organosilicon compounds of formula (II) each bear at their two ends the silicon-containing groupings (R₅O)_{c}(R₆)_{d}Si— and —Si(R₆′)_{d′}(OR₅′)_{c′}.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g, and h can independently of one another stand for the number 0 or 1, with the proviso that at least one of the radicals e, f, g, and h is different from 0. In other words, an organic silicon compound of formula (II) contains at least one grouping selected from the group including -(A)- and —[NR₇-(A′)]- and —[O-(A″)]- and —[NR₈-(A′″)]-.

In the two terminal structural units (R₅O)_{c}(R₆)_{d}Sii- and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from about 1 to about 3, and d stands for the integer 3−c. If c stands for the number 3, then d is equal to 0. If c stands for the number 2, then d is equal to 1. If c stands for the number 1, then d is equal to 2.

Analogously c′ stands for a whole number from about 1 to about 3, and d′ stands for the whole number 3−c′. If c′ stands for the number 3, then d′ is 0. If c′ stands for the number 2, then d′ is 1. If c′ stands for the number 1, then d′ is 2.

An extremely high anti-pollution effect of the agent for the treatment of a keratinous material could be obtained when the residues c and c′ both stand for the number 3. In this case d and d′ both stand for the number 0.

In another suitable one, the agent for treating a keratinous material includes at least one organic silicon compound of formula (II)

(R₅O)_{c}(R₆)_{d}Si-(A)_{c}-[NR₇-(A′)]_{f}-[O-(A″)]_{g}-[NR₈-(A′″)]ₕ-Si(R₆′)_{d′}(OR₅′)_{c′} (II),

where
R5 and R5′ independently represent a methyl group or an ethyl group,
c and c′ both stand for the number 3 and
d and d′ both stand for the number 0.

When c and c′ both represent the number 3 and d and d′ both represent the number 0, the organic silicon compounds correspond to formula (IIa)

(R₅O)₃Si-(A)ₑ-[NR₇-(A′)]_{f}-[O-(A″)]_{g}-[NR₈-(A′″)]ₕ-Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)ₑ- and -[NR7-(A′)]_{f}- and —[O-(A″)]_{g}- and -[NR8-(A′″)]ₕ- are in the middle part of the organic silicon compound of formula (II).

In this context, the presence of certain groupings has proven to be particularly beneficial in terms of increasing the “anti-pollution” effect. Particularly good results were obtained when at least two of the residues e, f, g, and h stand for the number 1. Especially suitable e and f both stand for the number 1. Furthermore, g and h both stand for the number 0.

When e and f are both 1 and g and h are both 0, the organic silicon compounds are represented by the formula (IIb)

(R₅O)_{c}(R₆)_{d}Si-(A)-[NR₇-(A′)]-Si(R₆′)_{d′}(OR₅′)_{c′} (IIb).

The radicals A, A′, A″, A′″ and A″″ independently represent a linear or branched two band C₁-C₂₀ alkylene group. In an exemplary embodiment the radicals A, A′, A″, A′″ and A″″ independently of one another represent a linear, two band C₁-C₂₀ alkylene group. Further in an exemplary embodiment the radicals A, A′, A″, A′″ and A″″ independently represent a linear two band C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

When the radical f represents the number 1, the organic silicon compound of formula (II) contains a structural grouping —[NR₇-(A′)]-.

When the radical h represents the number 1, the organic silicon compound of formula (II) contains a structural grouping —[NR₈-(A″)]-.

Wherein R₇ and R₇ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

-(A″″)-Si(R₆″)_{d}′(OR₅″)_{c}″ (III).

In an exemplary embodiment, R₇ and R₈ independently represent a hydrogen atom, a methyl group, a 2-hydroxyethyl group, a 2-alkenyl group, a 2-aminoethyl group or a group of formula (III).

When the radical f represents the number 1 and the radical h represents the number 0, the organic silicon compound contains the grouping [NR₇-(A′)] but not the grouping —[NR₈-(A″)]. If the radical R7 now stands for a grouping of the formula (III), the agent for treating a keratinous material contains an organic silicon compound with 3 reactive silane groups.

In another suitable one, the agent for treating a keratinous material includes at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
e and f both stand for the number 1,
g and h both stand for the number 0,
A and A′ independently represent a linear, two-band C₁-C₆ alkylene group and
R₇ represents a hydrogen atom, a methyl group, a 2-hydroxyethyl group, a 2-alkenyl group, a 2-aminoethyl group or a group of formula (III).

In another suitable embodiment, the composition for treating a keratinous material includes at least one organic silicon compound of the formula (II), wherein
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂), and
R₇ represents a hydrogen atom, a methyl group, a 2-hydroxyethyl group, a 2-alkenyl group, a 2-aminoethyl group or a group of formula (III).

Organic silicon compounds of formula (II) that are well suited for solving the problem are
3-(trimethoxysilyl)-N-[3-(trimethoxysilyl)propyl]-1-propanamine

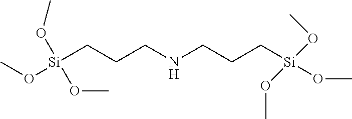

3-(Triethoxysilyl)-N-[3-(triethoxysilyl)propyl]-1-propanamine

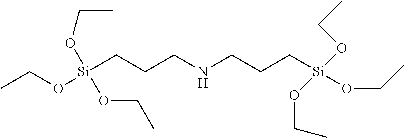 [SMILES: CCO[Si](CCCNCCC[Si](OCC)(OCC)OCC)(OCC)OCC]

N-methyl-3-(trimethoxysilyl)-N-[3-(trimethoxysilyl)propyl]-1-propanamine

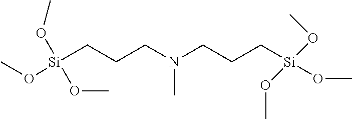

N-Methyl-3-(triethoxysilyl)-N-[3-(triethoxysilyl)propyl]-1-propanamine

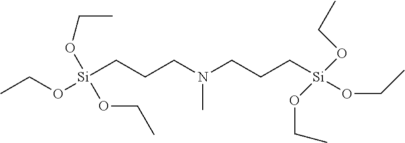 [SMILES: CCO[Si](CCCN(C)CCC[Si](OCC)(OCC)OCC)(OCC)OCC]

2-[Bis[3-(trimethoxysilyl)propyl]amino]-ethanol

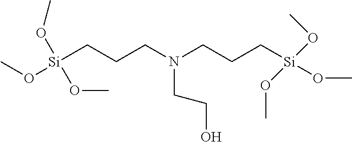

2-[bis[3-(triethoxysilyl)propyl]amino]ethanol

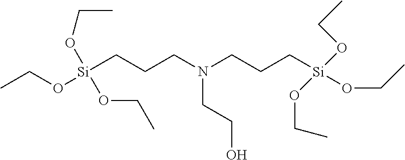 [SMILES: CCO[Si](CCCN(CCO)CCC[Si](OCC)(OCC)OCC)(OCC)OCC]

3-(Trimethoxysilyl)-N,N-bis[3-(trimethoxysilyl)propyl]-1-propanamine

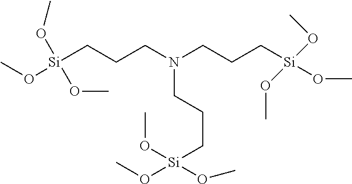

3-(Triethoxysilyl)-N,N-bis[3-(triethoxysilyl)propyl]-1-propanamine

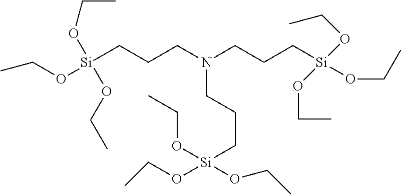 [SMILES: CCO[Si](CCCN(CCC[Si](OCC)(OCC)OCC)CCC[Si](OCC)(OCC)OCC)(OCC)OCC]

N1,N1-Bis[3-(trimethoxysilyl)propyl]-12-ethanediamine,

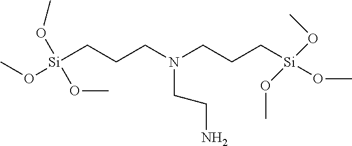

N1,N1-Bis[3-(triethoxysilyl)propyl]-1,2-ethanediamine,

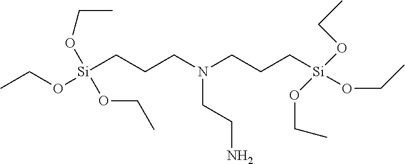 [SMILES: CCO[Si](CCCN(CCN)CCC[Si](OCC)(OCC)OCC)(OCC)OCC]

N,N-Bis[3-(trimethoxysilyl)propyl]-2-propen-1-amine

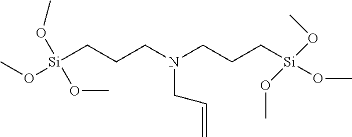

N,N-Bis[3-(triethoxysilyl)propyl]-2-propen-1-amine

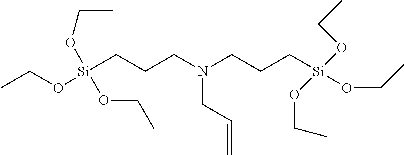 [SMILES: C=CCN(CCC[Si](OCC)(OCC)OCC)CCC[Si](OCC)(OCC)OCC]

The organic silicon compounds of formula (II) are commercially available.

Bis(trimethoxysilylpropyl)amine with the CAS number 82985-35-1 can be purchased from Sigma-Aldrich.

Bis[3-(triethoxysilyl)propyl]amine, also known as 3-(triethoxysilyl)-N-[3-(triethoxysilyl)propyl]-1-propanamine, with CAS number 13497-18-2 k can be purchased, for example, from Sigma-Aldrich or is commercially available under the product name Dynasylan 1122 from Evonik.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

It has also been found to be advantageous when the agent for treating a keratinous material applied to the hair contains at least one organic silicon compound of formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be referred to as silanes of the alkylalkoxysilane or alkylhydroxysilane type,

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a further suitable embodiment, the composition for treating a keratinous material contains, in addition to the organic silicon compound or compounds of formula (I), at least one further organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In a likewise suitable embodiment, the composition for treating a keratinous material contains, in addition to the organic silicon compound or compounds of the formula (II), at least one further organic silicon compound of the formula (IV) contains

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In another suitable embodiment, the composition for treating a keratinous material contains, in addition to the organic silicon compounds of the formula (I) and (II), at least one further organic silicon compound of the formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3−k.

In the organic silicon compounds of formula (IV), the radical R₉ represents a C₁-C₁₂ alkyl group. This C₁-C₁₂ alkyl group is saturated and can be linear or branched. In an exemplary embodiment R₉ stands for a linear C₁-C₈ alkyl group. In an exemplary embodiment R₉ stands for a methyl group, an ethyl group, an n-propyl group, an n-butyl group, an n-pentyl group, an n-hexyl group, an n-octyl group or an n-dodecyl group. For example, R₉ represents a methyl group, an ethyl group or an n-octyl group.

In the organic silicon compounds of formula (IV), the radical R₁₀ represents a hydrogen atom or a C₁-C₆ alkyl group. For example, R₁₀ represents a methyl group or an ethyl group.

In the organic silicon compounds of formula (IV), the radical R₁₁ represents a C₁-C₆ alkyl group. For example, R₁₁ represents a methyl group or an ethyl group.

Furthermore, k stands for a whole number from about 1 to about 3, and m stands for the whole number 3−k. If k stands for the number 3, then m is equal to 0. If k stands for the number 2, then m is equal to 1. If k stands for the number 1, then m is equal to 2.

An extremely high “anti-pollution” effect could be obtained if the agent for treating a keratinous material contains at least one organic silicon compound of formula (IV) in which the radical k represents the number 3. In this case the rest m stands for the number 0.

Organic silicon compounds of the formula (IV) that are particularly suitable for solving the problem are
Methyltrimethoxysilane

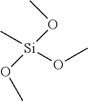

Methyltriethoxysilane

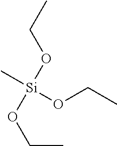

Ethyltrimethoxysilane

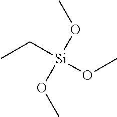

Ethyltriethoxysilane

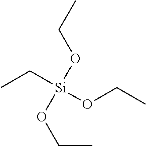

n-Hexyltrimethoxysilane

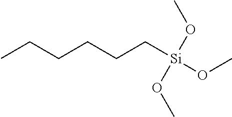

n-Hexyltriethoxysilane

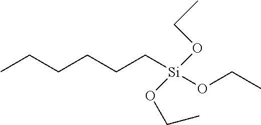 [SMILES: CCCCCC[Si](OCC)(OCC)OCC]

n-Octyltrimethoxysilane

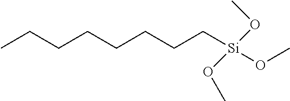

n-Octyltriethoxysilane

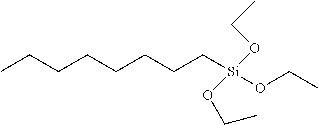 [SMILES: CCCCCCCC[Si](OCC)(OCC)OCC]

n-dodecyltrimethoxysilane and/or

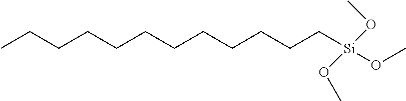 [SMILES: CCCCCCCCCCCC[Si](OC)(OC)OC]

n-Dodecyltriethoxysilane.

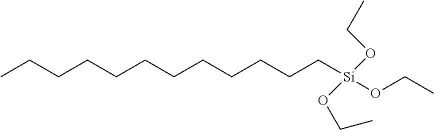 [SMILES: CCCCCCCCCCCC[Si](OCC)(OCC)OCC]

and propyltrimethoxysilane, propyltriethoxysilane, octadecyltrimethoxysilane and/or octadecyltriethoxysilane.

The organic silicon compounds described above are reactive compounds.

In this context, it has been found to be quite suitable if the agent contains as organic silicon compound (3-aminopropyl)triethoxysilane, i.e., an aminopropyltriethoxysilane (AMEO), and/or 3-(triethoxysilyl)-N-[3-(triethoxysilyl)propyl]-1-propanamine, i.e., a bis(triethoxysilylpropyl)amine.

According to suitable embodiments of the present disclosure, the organic silicon compound of formula (I), in particular the (3-aminopropyl)triethoxysilane, is present in an amount of from about 0.01 to about 10 wt. %, in an exemplary embodiment from about 0.02 to about 8-by weight, such as from about 0.05 to about 6-% by weight, in particular embodiments from about 0.1 to about 4-% by weight, based on the total weight of the cosmetic product, in the cosmetic product, and/or the organic silicon compound of the formula (II), in particular the 3-(triethoxysilyl)-N-[3-(triethoxysilyl)propyl]-1-propanamine, is present in an amount of from about 0.01 to about 10% by weight, in an exemplary embodiment from about 0.02 to about 9-by weight, such as from about 0.05 to about 8-% by weight, in particular embodiments from about 0.1 to about 7-%, by weight, based on the total weight of the cosmetic composition, in the cosmetic composition.

It was found that particularly stable and uniform films could be obtained on the keratinous material even when the agent contained two structurally different organic silicon compounds.

In a suitable embodiment, an agent exemplified includes at least one organic silicone compound of formula (I) and at least one organic silicone compound of formula (IV).

In an explicitly very particularly suitable embodiment, an agent exemplified contains at least one organic silicone compound of formula (I) selected from the group including (3-aminopropyl)triethoxysilane and (3-aminopropyl)trimethoxysilane, and additionally containing at least one organic silicone compound of formula (IV) selected from the group including methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In another suitable embodiment, an agent is exemplified in that the agent—contains, based on the total weight of the agent-:
about 0.5 to about 5 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
about 3.2 to about 10 wt. % of at least one second organic silicon compound selected from the group including methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

Even the addition of small amounts of water leads to hydrolysis in organic silicon compounds with at least one hydrolysable group. The hydrolysis products and/or organic silicon compounds having at least one hydroxy group may react with each other in a condensation reaction. For this reason, both the organosilicon compounds having at least one hydrolysable group and their hydrolysis and/or condensation products may be present in the composition. When organosilicon compounds having at least one hydroxyl group are used, both the organic silicon compounds having at least one hydroxyl group and their condensation products may be present in the composition.

A condensation product is understood to be a product formed by the reaction of at least two organic silicon compounds each having at least one hydroxyl group or hydrolysable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium shifts from monomeric organic silicon compounds to condensation product.

In the context of the present disclosure, figures in wt.-% are—unless otherwise stated—always based on the total weight of the cosmetic product.

As a second ingredient essential to the present disclosure, the cosmetic composition for treating a keratinous material contains, as a further component b), an antioxidant. In the course of the work leading to the present disclosure, it has been found that, to achieve a particularly good care and protective effect, it is particularly advantageous if the organic silicon compounds, for example (3-aminopropyl)triethoxysilane, i. e. (3-aminopropyl)triethoxysilane, i.e., an aminopropyltriethoxysilane (AMEO), or for example 3-(triethoxysilyl)-N-[3-(triethoxysilyl)propyl]-1-propanamine, i.e., a bis(triethoxysilylpropyl)amine, is combined with an antioxidant.

An antioxidant should first be understood as any agent that is a free radical scavenger. They include natural substances such as tocopherol and its derivatives as well as synthetic radical scavengers such as butylated hydroxyanisole (BHA), butylated hydroxytoluene (BHT) and alkyl gallates. In an exemplary embodiment, an antioxidant should be understood to mean a reducing agent, such as one that has an exceptionally low redox potential. For example, an antioxidant is to be understood as a reducing agent of redox potential +0.12 V and lower. Representatives of reducing agents with low redox potential are, for example. ascorbic acid (−0.04 V at pH 7 and 25° C.) and salts of sulfurous acid (+0.12 V at pH 7 and 25° C.), the redox potential being measured against the standard hydrogen electrode according to the usual standard.

It has been found that the combination of an antioxidant as understood herein, on the one hand, and the aminopropyltriethoxysilane (AMEO) and/or the bis(triethoxysilylpropyl)amine, on the other hand, is particularly powerful in terms of a conditioning treatment.

Suitable antioxidants are based on phenol derivatives or flavonoids with the following basic structure.

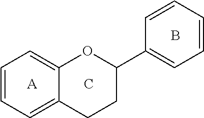

or flavonoid derivatives, especially rutin or sulfates of rutin. Furthermore, anthocyanins are suitable antioxidants, such as pelargonidin, cyanidin, peonidin, paeonidin, rosinidin, delphinidin, petunidin or malvidin. Other suitable antioxidants include vitamins or their derivatives. Suitable antioxidants are therefore one or more selected from the group including retinol, thiamine, riboflavin, niacin, nicotinamide, nicotinic acid, pantothenic acid, pyridoxine, pyridoxal, pyridoxamine, biotin, folic acid, cobalamin, cholecalciferol, tocopherol, phylloquinone, menaquinone, linolenic acid and amygdalin. Furthermore, carotenoids are suitable antioxidants.

According to a suitable embodiment, component b) comprising an antioxidant is used with a further skin moisturizing agent in the cosmetic composition. The further skin moisturizing agent is selected from the group including glycerin, urea, hyaluronic acid, silanol esters of hyaluronic acid, panthenol, taurine, ceramides, phytosterols, aloe vera extracts, creatine, creatinine, sodium hyaluronate, polysaccharides, biosaccharides gum-1, cucumber extracts, butylene glycol, propylene glycol, methyl propanediol, ethylhexyl glycerol, sorbitol, amino acids, glycine, glycine soy, histidine, tyrosine or tryptophan being particularly suitable amino acids, amino acid derivatives, natural betaine compounds, lactic acid, lactates, in particular sodium lactate, and/or ethylhexyloxyglycerol. In particular, the selection of these additional skin moisturizers increases the caring character of the cosmetic product.

According to a suitable embodiment of the present disclosure, component b) is present in the cosmetic composition in a total amount ranging from 0.1 to 10-% by weight, in an exemplary embodiment from 0.5 to 8-% by weight, such as from 1 to 6-% by weight.

In particular, the features for treating a keratinous material may include a features for cleaning a keratinous material, a features for maintaining a keratinous material, a features for maintaining and cleaning a keratinous material, and/or a features for temporarily reshaping a keratinous material.

In the following, further ingredients of the hair treatment products are described, which may be contained in the products in addition to the previously described mandatory ingredients.

It may be suitable that the agent for treating a keratinous material further includes from about 0.001 to about 20% by weight of at least one quaternary compound. This applies to agents for the care of a keratinous material and agents for the care and cleaning of a keratinous material.

It is suitable that the at least one quaternary compound is selected from at least one of the groups including:
i) of the monoalkylquats and/or
ii) the esterquats and/or
iii) of the quaternary imidazolines of formula (Tkat2),

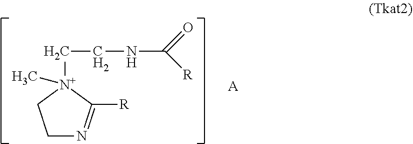

in which the radicals R independently of one another each represent a saturated or unsaturated, linear, or branched hydrocarbon radical having a chain length of 8 to 30 carbon atoms and A represents a physiologically tolerated anion, and/or
(iv) the amidoamines and/or cationized amidoamines and/or
v) Poly(methacryloyloxyethyltrimethylammonium compounds) and/or;
vi) quaternized cellulose derivatives, polyquaternium 10, polyquaternium-24, polyquaternium-27, polyquaternium-67, polyquaternium-72, and/or
vii) cationic alkyl polyglycosides and/or
viii) cationized honey and/or
ix) cationic guar derivatives and/or
x) Chitosan and/or
xi) polymeric dimethyldiallylammonium salts and copolymers thereof with esters and amides of acrylic acid and methacrylic acid, in particular polyquaternium-7 and/or
xii) Copolymers of vinylpyrrolidone with quaternized derivatives of dialkylaminoalkyl acrylate and methacrylate, especially polyquaternium-11 and/or
xiii) vinylpyrrolidone-vinylimidazolium methochloride copolymers, in particular polyquaternium-16 and/or
xiv) quaternized polyvinyl alcohol and/or
xv) Polyquaternium-74,
xvi) and mixtures thereof.

It is particularly suitable that the hair treatment composition contains a cationic hompolymer falling under the INCI designation polyquaternium-37 as quaternary compounds.

It may be suitable that the agent for treating a keratinous material further includes a firming compound, in an exemplary embodiment selected from the group including waxes, synthetic polymers, and mixtures thereof.

To meet the different requirements for agents for the treatment of a keratinous material in the form of an agent for the temporary reshaping of a keratinous material (=styling agent), many synthetic polymers have already been developed as strengthening compounds, which can be used in the agent for the treatment of a keratinous material. Alternatively, or complementarily, waxes are used as strengthening compounds. Ideally, the polymers and/or waxes, when applied to the keratinous material, result in a polymer film or sheet that, on the one hand, gives the hairstyle a strong hold, but, on the other hand, is sufficiently flexible not to break when stressed.

The synthetic polymers can be divided into cationic, anionic, nonionic, and amphoteric strengthening polymers.

Suitable synthetic polymers include, for example, polymers with the following INCI designations: Acrylamides/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/Ammonium Methacrylate Copolymer, Acrylates/t-Butyl Acrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopic Acid ntyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetone Acrylamide Copolymer, Ammonium-Acrylates/VA/Acrylates Copolymer, AMPD-Acrylates/Acrylamides Copolymer, AMPD-Acrylates/Acrylamides/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Cross polymer, Diphenyl Amodimethicone, Ethyl Ester of PVM/MA Copolymer, Hydrolyzed Wheat Protein/PVP Cross polymer, Isobutylene/Ethylmaleimide/Hydroxyethylmaleimide Copolymer, Isobutylene/MA Copolymer, Isobutylmethacrylate/Bis-hydroxypropyl Dimethicone Acrylate Copolymer, Isopropyl Ester of PVM/MA Acrylate Crosspolymer, Lauryl Methacrylate/Glycol Dimethacrylate Cross polymer, MEA-Sulphite, Methacrylic Acid/Sodium Acrylamidomethyl Propane Sulphonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophen-2, Polyquaternium-2 Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, 18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polquaternium-56 9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 polyglyce ryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylbiloxylsilicon VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers such as hydroxypropyl cellulose, hydroxyethyl cellulose and methyl hydroxypropyl cellulose are also suitable.

Also, homopolyacrylic acid (INCI: Carbomer), which is commercially available under the name Carbopol® in various forms, is suitable as a firming compound.

In an exemplary embodiment, the firming compound includes a vinylpyrrolidone-containing polymer. For example, the firming compound includes a polymer selected from the group including polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), and mixtures thereof.

Another suitable solidifying compound is octylacrylamide/acrylates/butylaminoethyl methacrylate copolymer (INCI), which is marketed under the name “Amphomer®” by Akzo Nobel.

Accordingly, it is particularly suitable that the firming compound includes a synthetic polymer selected from the group including polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI), and mixtures thereof.

According to further suitable embodiments of the present disclosure, the cosmetic composition includes at least one cationic surfactant as component d). This is for example a cationic surfactant of formula (V),

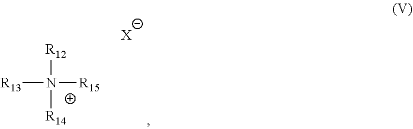

wherein
R₁₂, R₁₃, R₁₄ independently represent a C1-C6 alkyl group, a C2-C6 alkenyl group or a C2-C6 hydroxyalkyl group,
R₁₅ is a C8-C28 alkyl group, in an exemplary embodiment a C10-C22 alkyl group, and
X− represents a physiologically compatible anion,
and/or the cosmetic composition in an exemplary embodiment includes at least one cationic surfactant of the formula (VI),

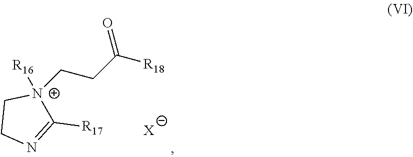

wherein
R₁₆ represents a C1-C6 alkyl group
R₁₇, R₁₈ independently represent a C7-C27 alkyl group, in an exemplary embodiment a C10-C22 alkyl group, and
X− represents a physiologically compatible anion,
and/or the cosmetic composition in an exemplary embodiment includes at least one cationic surfactant of the formula (VII),

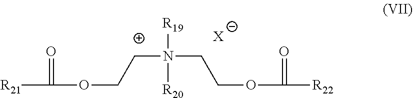

wherein
R₁₉, R₂₀ independently represent a C1-C6 alkyl group or a C2-C6 hydroxyalkyl group,
R₂₁, R₂₂ independently represent a C7-C27 alkyl group, in an exemplary embodiment a C10-C22 alkyl group, and
X− represents a physiologically compatible anion,
and/or the cosmetic composition in an exemplary embodiment includes at least one cationic surfactant of the formula (VIII),

NR₂₃R₂₄R₂₅ (VIII)

wherein
R₂₃, R₂₄ independently represent a C1-C6 alkyl group, a C2-C6 alkenyl group or a C2-C6 hydroxyalkyl group, and
R₂₅ represents a C8-C28 alkyl group, in an exemplary embodiment a C10-C22 alkyl group,

The cationic surfactants of formula (VIII) are amine derivatives, so-called pseudoquats. The organic radicals R₂₃, R₂₄ and R₂₅ are directly bonded to the nitrogen atom. In the acidic pH range, these are cationized, i.e., the nitrogen atom is then protonated. The physiologically compatible counterions are suitable as counterions. Steamidopropyl dimethylamine is particularly suitable among the cationic surfactants of formula (VIII).

According to a suitable embodiment of the present disclosure, the amount of cationic surfactant is about 0.1 to about 30-% by weight, in an exemplary embodiment from about 0.5 to about 20-% by weight, such as from about 1 to about 10-% by weight, based on the total weight of the cosmetic composition.

According to a suitable embodiment of the present disclosure, the cationic surfactant includes a hydrophobic head group with a cationic charge and one or two hydrophobic end portions, wherein the hydrophobic end portion(s) represent straight-chain or branched, saturated or mono- or polyunsaturated alkyl groups, in an exemplary embodiment having a chain length of C6 to C30, such as C8 to C26, for example C10 to C22. According to another suitable embodiment, the cationic surfactant has an ester function, an ether function, a ketone function, an alcohol function, or an amide function.

According to suitable embodiments of the present disclosure, one or more anionic surfactants are included in the cosmetic composition, in an exemplary embodiment selected from the group including:
straight-chain or branched, saturated or mono- or polyunsaturated alkyl sulfonates containing 8 to 24, in an exemplary embodiment 12 to 22, such as 16 to 18 carbon atoms,
- linear alpha-olefin sulfonates with 8 to 24, in an exemplary embodiment 12 to 22, such as 16 to 18 C atoms,
Alkyl sulfates and alkyl polyglycol ether sulfates of the formula R₉—O—(CH₂—CH₂O)ₙ—SO₃X, in which R₉ is in an exemplary embodiment a straight-chain or branched, saturated or mono- or polyunsaturated alkyl or alkenyl radical having 8 to 24, in an exemplary embodiment 12 to 22, such as 16 to 18 carbon atoms, n is 0 or 1 to 12, such as 2 to 4, and X is an alkali metal or alkaline earth metal ion or protonated triethanolamine or the ammonium ion,
straight-chain or branched, saturated or mono- or polyunsaturated alkylcarboxylic acids containing 8 to 24, in an exemplary embodiment 12 to 22, such as 16 to 18 carbon atoms,
- straight-chain or branched, saturated or mono- or polyunsaturated alkyl phosphates containing 8 to 24, in an exemplary embodiment 12 to 22, such as 16 to 18 carbon atoms,
Alkyl isethionate whose alkyl group is selected from a branched or unbranched C₆ to C₂₂, in an exemplary embodiment C₁₀ to C₁₈, such as C₁₂ to C₁₆ alkyl group, in particular sodium cocoyl isethionate,
Alkyl glycoside carboxylic acids whose alkyl group is selected from a branched or unbranched C₆ to C₂₂, in an exemplary embodiment C₁₀ to C₁₈, such as C₁₂ to C₁₆ alkyl group,
Alkyl sulfosuccinates, the two alkyl groups of which are selected from identical or different, branched, or unbranched C₂ to C₁₂, in an exemplary embodiment C₄ to C₁₀, such as C₆ to C₈ alkyl groups,
Alkyl taurates, the alkyl group of which is selected from a branched or unbranched C₆ to C₂₂, in an exemplary embodiment C₁₀ to C₁₈, such as C₁₂ to C₁₆ alkyl group,
Alkyl sarcosinates whose alkyl group is selected from a branched or unbranched C₆ to C₂₂, in an exemplary embodiment C₁₀ to C₁₈, such as C₁₂ to C₁₆ alkyl group,
Sulfonates of unsaturated fatty acids with 8 to 24, in an exemplary embodiment 12 to 22, such as 16 to 18 C atoms and 1 to 6 double bonds,
   wherein the counterion of the anionic surfactant is an alkali or alkaline earth metal ion or a protonated triethanolamine or the ammonium ion.

Particularly suitable anionic surfactants are straight-chain or branched alkyl ether sulfates containing an alkyl radical with 8 to 18 and with 10 to 16 carbon atoms and 1 to 6 and 2 to 4 ethylene oxide units. In particular embodiments, the surfactant mixture of anionic and amphoteric/zwitterionic surfactants contains sodium lauryl ether sulfate (INCI: sodium laureth sulfate) and in particular embodiments sodium lauryl ether sulfate with 2 ethylene oxide units.

Amphoteric surfactants, also known as zwitterionic surfactants, are surface-active compounds that contain at least one quaternary ammonium group and at least one —COO— or —SO₃— group in the molecule. Amphoteric/zwitterionic surfactants also include surface-active compounds which, in addition to a C₈-C₂₄ alkyl or acyl group, contain at least one free amino group and at least one —COOH or —SO₃H group and can form internal salts.

According to a suitable embodiment of the present disclosure, the amphoteric surfactants in the cosmetic composition are selected from the group including:
Alkyl betaine comprising at least one saturated or unsaturated, branched, or unbranched C₆ to C₂₂, in an exemplary embodiment C₁₀ to C₁₈, such as C₁₂ to C₁₆ alkyl group,
Alkyl amphodiacetate or alkyl amphodiacetate comprising a saturated or unsaturated, branched, or unbranched C₆ to C₂₂, in an exemplary embodiment C₁₀ to C₁₈, such as C₁₂ to C₁₆ alkyl group, with an alkali or alkaline earth metal counterion, and
Alkylamidopropyl betaine comprising at least one saturated or unsaturated, branched, or unbranched C₆ to C₂₂, in an exemplary embodiment C₁₀ to C₁₈, such as C₁₂ to C₁₆ alkyl group.

Particularly suitable amphoteric/zwitterionic surfactants include those known under the INCI designation cocamidopropyl betaine and disodium cocoamphodiacetate.

According to a suitable embodiment of the present disclosure, the nonionic surfactant is selected from the group including:
Alkylglucamide comprising a saturated or unsaturated, branched, or unbranched C₆ to C₂₂, in an exemplary embodiment C₁₀ to C₁₈, such as C₁₂ to C₁₆ alkyl group,
Alkyl fructoside comprising a saturated or unsaturated, branched, or unbranched C₆ to C₂₂, in an exemplary embodiment C₁₀ to C₁₈, such as C₁₂ to C₁₆ alkyl group,
An alkyl glucoside comprising a saturated or unsaturated, branched, or unbranched C₆ to C₂₂, in an exemplary embodiment C₁₀ to C₁₈, such as C₁₂ to C₁₆ alkyl group,
Alkyl alcohol alkoxylate of the formula R₁₀(OR₁₁)ₘOH, in which R₁₀ represents a linear or branched C₆-C₂₂, in an exemplary embodiment C₁₀-C₁₈, such as C₁₂-C₁₆ alkyl group, R₁₁ represents a C₂-C₄, in an exemplary embodiment a C₂ alkyl group, and m represents 1 to 10, in an exemplary embodiment 2 to 6, such as 2 to 6, and
Alkyl esters of the formula R₁₂COOR₁₃, in which R₁₂ represents a linear or branched C₆ to C₂₂, in an exemplary embodiment C₁₀ to C₁₈, such as C₁₂ to C₁₆ alkyl group, R₁₃ represents a C₁ to C₄, in an exemplary embodiment a C₂ alkyl group.

According to a suitable embodiment of the present disclosure, the cosmetic composition contains two structurally different surfactants. It is particularly suitable that the cosmetic agent contains two structurally different surfactants from one another, in an exemplary embodiment the cosmetic agent contains two structurally different cationic surfactants from one another, or the cosmetic agent contains a cationic surfactant and a nonionic surfactant.

The cosmetic composition may contain, in addition or as an alternative to a synthetic polymer, at least one natural or synthetic wax having a melting point above 37° C. as a firming compound.

Natural or synthetic waxes can be solid kerosenes or isoparaffins, vegetable waxes such as candelilla wax, carnauba wax, esparto grass wax, Japan wax, cork wax, sugar cane wax, ouricury wax, montan wax, sunflower wax, fruit waxes and animal waxes such as beeswaxes and other insect waxes, Whale wax, shellac wax, wool wax and brushing grease, furthermore mineral waxes, such as ceresin and ozokerite or petrochemical waxes, such as petrolatum, kerosene waxes, microwaxes of polyethylene or polypropylene and polyethylene glycol waxes can be used. It may be advantageous to use hydrogenated or cured waxes. Chemically modified waxes, in particular hard waxes such as montan ester waxes, sasol waxes and hydrogenated jojoba waxes, can also be used.

Furthermore, in addition to the mandatory components, the triglycerides of saturated and optionally hydroxylated C₁₆-30 fatty acids, such as hydrogenated triglyceride fats (hydrogenated palm oil, hydrogenated coconut oil, hydrogenated castor oil), glyceryl tribehenate or glyceryl tri-12-hydroxystearate are suitable in the cosmetic products.

The wax components can also be selected from the group of esters of saturated, unbranched alkanecarboxylic acids having a chain length of 22 to 44 carbon atoms and saturated, unbranched alcohols having a chain length of 22 to 44 carbon atoms, provided that the wax component or the totality of wax components are solid at room temperature. Silicone waxes, for example stearyltrimethylsilane/stearyl alcohol, may also be beneficial.

Natural, chemically modified, and synthetic waxes can be used alone or in combination. Thus, several waxes can also be used. Furthermore, several wax mixtures, possibly mixed with other additives, are also commercially available. The products sold under the designations “Special Wax 7686 OE” (a mixture of cetyl palmitate, beeswax, microcrystalline wax and polyethylene with a melting range of about 73-75° C.; manufacturer: Kahl & Co), Polywax® GP 200 (a mixture of stearyl alcohol and polyethylene glycol stearate with a melting point of about 47-51° C.; manufacturer: Croda) and “Softceresin® FL 400” (a vaseline/vaseline oil/wax mixture with a melting point of about 50-54° C.; manufacturer: Parafluid Mineral Oil Company) are examples of mixtures that can be used.

In an exemplary embodiment, the wax is selected from carnauba wax (INCI: Copernicia Cerifera Cera) Bienenwachs (INCI: Beeswax), petrolatum (INCI), microcrystalline wax and especially mixtures thereof.

Suitable blends include the combination of carnauba wax (INCI: Copernicia Cerifera Cera), petrolatum and microcrystalline wax or the combination of beeswax (INCI: Beeswax) and petrolatum.

The wax or wax components should be solid at 25° C. and should melt in the range >37° C.

The composition for treating a keratinous material in an exemplary embodiment contains the firming compound in a total amount of about 0.5 to about 50% by weight, in an exemplary embodiment from about 1 to about 40% by weight, such as from about 1.5 to about 30% by weight, even such as from about 2 to about 25% by weight, based on the total weight of the cosmetic composition.

Other suitable ingredients include nonionic polymers, anionic polymers, waxes, protein hydrolysates, amino acids, oligopetides, vitamin precursors, betaines, bioquinones, purine (derivatives), plant extracts, silicones, ester oils, UV light protection filters, structuring agents, thickeners, electrolytes, pH-adjusting agents, swelling agents, colorants, anti-dandruff agents, complexing agents, opacifiers, pearlescent agents, pigments, stabilizing agents, propellants, perfume oils and/or preservatives.

In suitable embodiments 1 to 576, suitable organic silicon compounds are combined with suitable antioxidants in a cosmetic composition as contemplated herein.

| | Silane compound | other ingredient |
|---|---|---|
| 1 | -(3-Aminopropyl)trimethoxysilane | Retinol |
| 2 | (3-Aminopropyl)triethoxysilan | Retinol |
| 3 | -(2-Aminoethyl)trimethoxysilane | Retinol |
| 4 | -(2-Aminoethyl)triethoxysilane | Retinol |
| 5 | -(3-Dimethylaminopropyl)trimethoxysilane | Retinol |
| 6 | (3-Dimethylaminopropyl)triethoxysilane | Retinol |
| 7 | (2-Dimethylaminoethyl)trimethoxysilane | Retinol |
| 8 | (2-Dimethylaminoethyl)triethoxysilane | Retinol |
| 9 | 3-(trimethoxysilyl)-N-[3-(trimethoxysilyl)propyl]-1-propanamine | Retinol |
| 10 | 3-(Triethoxysilyl)-N-[3-(triethoxysilyl)propyl]-1-propanamine | Retinol |
| 11 | N-methyl-3-(trimethoxysilyl)-N-[3-(trimethoxysilyl)propyl]-1-propanamine | Retinol |
| 12 | N-Methyl-3-(triethoxysilyl)-N-[3-(triethoxysilyl)propyl]-1-propanamine | Retinol |
| 13 | 2-[Bis[3-(trimethoxysilyl)propyl]amino]-ethanol | Retinol |
| 14 | 2-[bis[3-(triethoxysilyl)propyl]amino]ethanol | Retinol |
| 15 | 3-(Trimethoxysilyl)-N,N-bis[3-(trimethoxysilyl)propyl]-1-propanamine | Retinol |
| 16 | 3-(Triethoxysilyl)-N,N-bis[3-(triethoxysilyl)propyl]-1-propanamine | Retinol |
| 17 | N1,N1-Bis[3-(trimethoxysilyl)propyl]-1,2-ethanediamine | Retinol |
| 18 | N1,N1-Bis[3-(triethoxysilyl)propyl]-1,2-ethanediamine | Retinol |
| 19 | N,N-Bis[3-(trimethoxysilyl)propyl]-2-propen-1-amine | Retinol |
| 20 | N,N-Bis[3-(triethoxysilyl)propyl]-2-propen-1-amine | Retinol |
| 21 | Methyltrimethoxysilane | Retinol |
| 22 | Methyltriethoxysilane | Retinol |
| 23 | Ethyltrimethoxysilane | Retinol |
| 24 | Ethyltriethoxysilane | Retinol |
| 25 | Octyltrimethoxysilane | Retinol |
| 26 | Octyltriethoxysilane | Retinol |
| 27 | Dodecyltrimethoxysilane | Retinol |
| 28 | Dodecyltriethoxysilane | Retinol |
| 29 | -(3-Aminopropyl)trimethoxysilane | Thiamine |
| 30 | (3-Aminopropyl)triethoxysilan | Thiamine |
| 31 | -(2-Aminoethyl)trimethoxysilane | Thiamine |
| 32 | -(2-Aminoethyl)triethoxysilane | Thiamine |
| 33 | -(3-Dimethylaminopropyl)trimethoxysilane | Thiamine |
| 34 | (3-Dimethylaminopropyl)triethoxysilane | Thiamine |
| 35 | (2-Dimethylaminoethyl)trimethoxysilane | Thiamine |
| 36 | (2-Dimethylaminoethyl)triethoxysilane | Thiamine |
| 37 | 3-(trimethoxysilyl)-N-[3-(trimethoxysilyl)propyl]-1-propanamine | Thiamine |
| 38 | 3-(Triethoxysilyl)-N-[3-(triethoxysilyl)propyl]-1-propanamine | Thiamine |
| 39 | N-methyl-3-(trimethoxysilyl)-N-[3-(trimethoxysilyl)propyl]-1-propanamine | Thiamine |
| 40 | N-Methyl-3-(triethoxysilyl)-N-[3-(triethoxysilyl)propyl]-1-propanamine | Thiamine |
| 41 | 2-[Bis[3-(trimethoxysilyl)propyl]amino]-ethanol | Thiamine |
| 42 | 2-[bis[3-(triethoxysilyl)propyl]amino]ethanol | Thiamine |
| 43 | 3-(Trimethoxysilyl)-N,N-bis[3-(trimethoxysilyl)propyl]-1-propanamine | Thiamine |
| 44 | 3-(Triethoxysilyl)-N,N-bis[3-(triethoxysilyl)propyl]-1-propanamine | Thiamine |
| 45 | N1,N1-Bis[3-(trimethoxysilyl)propyl]-1,2-ethanediamine | Thiamine |
| 46 | N1,N1-Bis[3-(triethoxysilyl)propyl]-1,2-ethanediamine | Thiamine |
| 47 | N,N-Bis[3-(trimethoxysilyl)propyl]-2-propen-1-amine | Thiamine |
| 48 | N,N-Bis[3-(triethoxysilyl)propyl]-2-propen-1-amine | Thiamine |
| 49 | Methyltrimethoxysilane | Thiamine |
| 50 | Methyltriethoxysilane | Thiamine |
| 51 | Ethyltrimethoxysilane | Thiamine |
| 52 | Ethyltriethoxysilane | Thiamine |
| 53 | Octyltrimethoxysilane | Thiamine |
| 54 | Octyltriethoxysilane | Thiamine |
| 55 | Dodecyltrimethoxysilane | Thiamine |
| 56 | Dodecyltriethoxysilane | Thiamine |
| 57 | -(3-Aminopropyl)trimethoxysilane | Riboflavin |
| 58 | (3-Aminopropyl)triethoxysilan | Riboflavin |
| 59 | -(2-Aminoethyl)trimethoxysilane | Riboflavin |
| 60 | -(2-Aminoethyl)triethoxysilane | Riboflavin |
| 61 | -(3-Dimethylaminopropyl)trimethoxysilane | Riboflavin |
| 62 | (3-Dimethylaminopropyl)triethoxysilane | Riboflavin |
| 63 | (2-Dimethylaminoethyl)trimethoxysilane | Riboflavin |
| 64 | (2-Dimethylaminoethyl)triethoxysilane | Riboflavin |
| 65 | 3-(trimethoxysilyl)-N-[3-(trimethoxysilyl)propyl]-1-propanamine | Riboflavin |
| 66 | 3-(Triethoxysilyl)-N-[3-(triethoxysilyl)propyl]-1-propanamine | Riboflavin |
| 67 | N-methyl-3-(trimethoxysilyl)-N-[3-(trimethoxysilyl)propyl]-1-propanamine | Riboflavin |
| 68 | N-Methyl-3-(triethoxysilyl)-N-[3-(triethoxysilyl)propyl]-1-propanamine | Riboflavin |
| 69 | 2-[Bis[3-(trimethoxysilyl)propyl]amino]-ethanol | Riboflavin |
| 70 | 2-[bis[3-(triethoxysilyl)propyl]amino]ethanol | Riboflavin |
| 71 | 3-(Trimethoxysilyl)-N,N-bis[3-(trimethoxysilyl)propyl]-1-propanamine | Riboflavin |
| 72 | 3-(Triethoxysilyl)-N,N-bis[3-(triethoxysilyl)propyl]-1-propanamine | Riboflavin |
| 73 | N1,N1-Bis[3-(trimethoxysilyl)propyl]-1,2-ethanediamine | Riboflavin |
| 74 | N1,N1-Bis[3-(triethoxysilyl)propyl]-1,2-ethanediamine | Riboflavin |
| 75 | N,N-Bis[3-(trimethoxysilyl)propyl]-2-propen-1-amine | Riboflavin |
| 76 | N,N-Bis[3-(triethoxysilyl)propyl]-2-propen-1-amine | Riboflavin |
| 77 | Methyltrimethoxysilane | Riboflavin |
| 78 | Methyltriethoxysilane | Riboflavin |
| 79 | Ethyltrimethoxysilane | Riboflavin |
| 80 | Ethyltriethoxysilane | Riboflavin |
| 81 | Octyltrimethoxysilane | Riboflavin |
| 82 | Octyltriethoxysilane | Riboflavin |
| 83 | Dodecyltrimethoxysilane | Riboflavin |
| 84 | Dodecyltriethoxysilane | Riboflavin |
| 85 | -(3-Aminopropyl)trimethoxysilane | Niacin |
| 86 | (3-Aminopropyl)triethoxysilan | Niacin |
| 87 | -(2-Aminoethyl)trimethoxysilane | Niacin |
| 88 | -(2-Aminoethyl)triethoxysilane | Niacin |
| 89 | -(3-Dimethylaminopropyl)trimethoxysilane | Niacin |
| 90 | (3-Dimethylaminopropyl)triethoxysilane | Niacin |
| 91 | (2-Dimethylaminoethyl)trimethoxysilane | Niacin |
| 92 | (2-Dimethylaminoethyl)triethoxysilane | Niacin |
| 93 | 3-(trimethoxysilyl)-N-[3-(trimethoxysilyl)propyl]-1-propanamine | Niacin |
| 94 | 3-(Triethoxysilyl)-N-[3-(triethoxysilyl)propyl]-1-propanamine | Niacin |
| 95 | N-methyl-3-(trimethoxysilyl)-N-[3-(trimethoxysilyl)propyl]-1-propanamine | Niacin |
| 96 | N-Methyl-3-(triethoxysilyl)-N-[3-(triethoxysilyl)propyl]-1-propanamine | Niacin |
| 97 | 2-[Bis[3-(trimethoxysilyl)propyl]amino]-ethanol | Niacin |
| 98 | 2-[bis[3-(triethoxysilyl)propyl]amino]ethanol | Niacin |
| 99 | 3-(Trimethoxysilyl)-N,N-bis[3-(trimethoxysilyl)propyl]-1-propanamine | Niacin |
| 100 | 3-(Triethoxysilyl)-N,N-bis[3-(triethoxysilyl)propyl]-1-propanamine | Niacin |
| 101 | N1,N1-Bis[3-(trimethoxysilyl)propyl]-1,2-ethanediamine | Niacin |
| 102 | N1,N1-Bis[3-(triethoxysilyl)propyl]-1,2-ethanediamine | Niacin |
| 103 | N,N-Bis[3-(trimethoxysilyl)propyl]-2-propen-1-amine | Niacin |
| 104 | N,N-Bis[3-(triethoxysilyl)propyl]-2-propen-1-amine | Niacin |
| 105 | Methyltrimethoxysilane | Niacin |
| 106 | Methyltriethoxysilane | Niacin |
| 107 | Ethyltrimethoxysilane | Niacin |
| 108 | Ethyltriethoxysilane | Niacin |
| 109 | Octyltrimethoxysilane | Niacin |
| 110 | Octyltriethoxysilane | Niacin |
| 111 | Dodecyltrimethoxysilane | Niacin |
| 112 | Dodecyltriethoxysilane | Niacin |
| 113 | -(3-Aminopropyl)trimethoxysilane | Pantothenic acid |
| 114 | (3-Aminopropyl)triethoxysilane | Pantothenic acid |
| 115 | -(2-Aminoethyl)trimethoxysilane | Pantothenic acid |
| 116 | -(2-Aminoethyl)triethoxysilane | Pantothenic acid |
| 117 | -(3-Dimethylaminopropyl)trimethoxysilane | Pantothenic acid |
| 118 | (3-Dimethylaminopropyl)triethoxysilane | Pantothenic acid |
| 119 | (2-Dimethylaminoethyl)trimethoxysilane | Pantothenic acid |
| 120 | (2-Dimethylaminoethyl)triethoxysilane | Pantothenic acid |
| 121 | 3-(trimethoxysilyl)-N-[3-(trimethoxysilyl)propyl]-1-propanamine | Pantothenic acid |
| 122 | 3-(Triethoxysilyl)-N-[3-(triethoxysilyl)propyl]-1-propanamine | Pantothenic acid |
| 123 | N-methyl-3-(trimethoxysilyl)-N-[3-(trimethoxysilyl)propyl]-1-propanamine | Pantothenic acid |
| 124 | N-Methyl-3-(triethoxysilyl)-N-[3-(triethoxysilyl)propyl]-1-propanamine | Pantothenic acid |
| 125 | 2-[Bis[3-(trimethoxysilyl)propyl]amino]-ethanol | Pantothenic acid |
| 126 | 2-[bis[3-(triethoxysilyl)propyl]amino]ethanol | Pantothenic acid |
| 127 | 3-(Trimethoxysilyl)-N,N-bis[3-(trimethoxysilyl)propyl]-1-propanamine | Pantothenic acid |
| 128 | 3-(Triethoxysilyl)-N,N-bis[3-(triethoxysilyl)propyl]-1-propanamine | Pantothenic acid |
| 129 | N1,N1-Bis[3-(trimethoxysilyl)propyl]-1,2-ethanediamine | Pantothenic acid |
| 130 | N1,N1-Bis[3-(triethoxysilyl)propyl]-1,2-ethanediamine | Pantothenic acid |
| 131 | N,N-Bis[3-(trimethoxysilyl)propyl]-2-propen-1-amine | Pantothenic acid |
| 132 | N,N-Bis[3-(triethoxysilyl)propyl]-2-propen-1-amine | Pantothenic acid |
| 133 | Methyltrimethoxysilane | Pantothenic acid |
| 134 | Methyltriethoxysilane | Pantothenic acid |
| 135 | Ethyltrimethoxysilane | Pantothenic acid |
| 136 | Ethyltriethoxysilane | Pantothenic acid |
| 137 | Octyltrimethoxysilane | Pantothenic acid |
| 138 | Octyltriethoxysilane | Pantothenic acid |
| 139 | Dodecyltrimethoxysilane | Pantothenic acid |
| 140 | Dodecyltriethoxysilane | Pantothenic acid |
| 141 | -(3-Aminopropyl)trimethoxysilane | Pyridoxine |
| 142 | (3-Aminopropyl)triethoxysilan | Pyridoxine |
| 143 | -(2-Aminoethyl)trimethoxysilane | Pyridoxine |
| 144 | -(2-Aminoethyl)triethoxysilane | Pyridoxine |
| 145 | -(3-Dimethylaminopropyl)trimethoxysilane | Pyridoxine |
| 146 | (3-Dimethylaminopropyl)triethoxysilane | Pyridoxine |
| 147 | (2-Dimethylaminoethyl)trimethoxysilane | Pyridoxine |
| 148 | (2-Dimethylaminoethyl)triethoxysilane | Pyridoxine |
| 149 | 3-(trimethoxysilyl)-N-[3-(trimethoxysilyl)propyl]-1-propanamine | Pyridoxine |
| 150 | 3-(Triethoxysilyl)-N-[3-(triethoxysilyl)propyl]-1-propanamine | Pyridoxine |
| 151 | N-methyl-3-(trimethoxysilyl)-N-[3-(trimethoxysilyl)propyl]-1-propanamine | Pyridoxine |
| 152 | N-Methyl-3-(triethoxysilyl)-N-[3-(triethoxysilyl)propyl]-1-propanamine | Pyridoxine |
| 153 | 2-[Bis[3-(trimethoxysilyl)propyl]amino]-ethanol | Pyridoxine |
| 154 | 2-[bis[3-(triethoxysilyl)propyl]amino]ethanol | Pyridoxine |
| 155 | 3-(Trimethoxysilyl)-N,N-bis[3-(trimethoxysilyl)propyl]-1-propanamine | Pyridoxine |
| 156 | 3-(Triethoxysilyl)-N,N-bis[3-(triethoxysilyl)propyl]-1-propanamine | Pyridoxine |
| 157 | N1,N1-Bis[3-(trimethoxysilyl)propyl]-1,2-ethanediamine | Pyridoxine |
| 158 | N1,N1-Bis[3-(triethoxysilyl)propyl]-1,2-ethanediamine | Pyridoxine |
| 159 | N,N-Bis[3-(trimethoxysilyl)propyl]-2-propen-1-amine | Pyridoxine |
| 160 | N,N-Bis[3-(triethoxysilyl)propyl]-2-propen-1-amine | Pyridoxine |
| 161 | Methyltrimethoxysilane | Pyridoxine |
| 162 | Methyltriethoxysilane | Pyridoxine |
| 163 | Ethyltrimethoxysilane | Pyridoxine |
| 164 | Ethyltriethoxysilane | Pyridoxine |
| 165 | Octyltrimethoxysilane | Pyridoxine |
| 166 | Octyltriethoxysilane | Pyridoxine |
| 167 | Dodecyltrimethoxysilane | Pyridoxine |
| 168 | Dodecyltriethoxysilane | Pyridoxine |
| 169 | -(3-Aminopropyl)trimethoxysilane | Biotin |
| 170 | (3-Aminopropyl)triethoxysilan | Biotin |
| 171 | -(2-Aminoethyl)trimethoxysilane | Biotin |
| 172 | -(2-Aminoethyl)triethoxysilane | Biotin |
| 173 | -(3-Dimethylaminopropyl)trimethoxysilane | Biotin |
| 174 | (3-Dimethylaminopropyl)triethoxysilane | Biotin |
| 175 | (2-Dimethylaminoethyl)trimethoxysilane | Biotin |
| 176 | (2-Dimethylaminoethyl)triethoxysilane | Biotin |
| 177 | 3-(trimethoxysilyl)-N-[3-(trimethoxysilyl)propyl]-1-propanamine | Biotin |
| 178 | 3-(Triethoxysilyl)-N-[3-(triethoxysilyl)propyl]-1-propanamine | Biotin |
| 179 | N-methyl-3-(trimethoxysilyl)-N-[3-(trimethoxysilyl)propyl]-1-propanamine | Biotin |
| 180 | N-Methyl-3-(triethoxysilyl)-N-[3-(triethoxysilyl)propyl]-1-propanamine | Biotin |
| 181 | 2-[Bis[3-(trimethoxysilyl)propyl]amino]-ethanol | Biotin |
| 182 | 2-[bis[3-(triethoxysilyl)propyl]amino]ethanol | Biotin |
| 183 | 3-(Trimethoxysilyl)-N,N-bis[3-(trimethoxysilyl)propyl]-1-propanamine | Biotin |
| 184 | 3-(Triethoxysilyl)-N,N-bis[3-(triethoxysilyl)propyl]-1-propanamine | Biotin |
| 185 | N1,N1-Bis[3-(trimethoxysilyl)propyl]-1,2-ethanediamine | Biotin |
| 186 | N1,N1-Bis[3-(triethoxysilyl)propyl]-1,2-ethanediamine | Biotin |
| 187 | N,N-Bis[3-(trimethoxysilyl)propyl]-2-propen-1-amine | Biotin |
| 188 | N,N-Bis[3-(triethoxysilyl)propyl]-2-propen-1-amine | Biotin |
| 189 | Methyltrimethoxysilane | Biotin |
| 190 | Methyltriethoxysilane | Biotin |
| 191 | Ethyltrimethoxysilane | Biotin |
| 192 | Ethyltriethoxysilane | Biotin |
| 193 | Octyltrimethoxysilane | Biotin |
| 194 | Octyltriethoxysilane | Biotin |
| 195 | Dodecyltrimethoxysilane | Biotin |
| 196 | Dodecyltriethoxysilane | Biotin |
| 197 | -(3-Aminopropyl)trimethoxysilane | Folic acid |
| 198 | (3-Aminopropyl)triethoxysilan | Folic acid |
| 199 | -(2-Aminoethyl)trimethoxysilane | Folic acid |
| 200 | -(2-Aminoethyl)triethoxysilane | Folic acid |
| 201 | -(3-Dimethylaminopropyl)trimethoxysilane | Folic acid |
| 202 | (3-Dimethylaminopropyl)triethoxysilane | Folic acid |
| 203 | (2-Dimethylaminoethyl)trimethoxysilane | Folic acid |
| 204 | (2-Dimethylaminoethyl)triethoxysilane | Folic acid |
| 205 | 3-(trimethoxysilyl)-N-[3-(trimethoxysilyl)propyl]-1-propanamine | Folic acid |
| 206 | 3-(Triethoxysilyl)-N-[3-(triethoxysilyl)propyl]-1-propanamine | Folic acid |
| 207 | N-methyl-3-(trimethoxysilyl)-N-[3-(trimethoxysilyl)propyl]-1-propanamine | Folic acid |
| 208 | N-Methyl-3-(triethoxysilyl)-N-[3-(triethoxysilyl)propyl]-1-propanamine | Folic acid |
| 209 | 2-[Bis[3-(trimethoxysilyl)propyl]amino]-ethanol | Folic acid |
| 210 | 2-[bis[3-(triethoxysilyl)propyl]amino]ethanol | Folic acid |
| 211 | 3-(Trimethoxysilyl)-N,N-bis[3-(trimethoxysilyl)propyl]-1-propanamine | Folic acid |
| 212 | 3-(Triethoxysilyl)-N,N-bis[3-(triethoxysilyl)propyl]-1-propanamine | Folic acid |
| 213 | N1,N1-Bis[3-(trimethoxysilyl)propyl]-1,2-ethanediamine | Folic acid |
| 214 | N1,N1-Bis[3-(triethoxysilyl)propyl]-1,2-ethanediamine | Folic acid |
| 215 | N,N-Bis[3-(trimethoxysilyl)propyl]-2-propen-1-amine | Folic acid |
| 216 | N,N-Bis[3-(triethoxysilyl)propyl]-2-propen-1-amine | Folic acid |
| 217 | Methyltrimethoxysilane | Folic acid |
| 218 | Methyltriethoxysilane | Folic acid |
| 219 | Ethyltrimethoxysilane | Folic acid |
| 220 | Ethyltriethoxysilane | Folic acid |
| 221 | Octyltrimethoxysilane | Folic acid |
| 222 | Octyltriethoxysilane | Folic acid |
| 223 | Dodecyltrimethoxysilane | Folic acid |
| 224 | Dodecyltriethoxysilane | Folic acid |
| 225 | -(3-Ammopropyl)trimethoxysilane | Cobalamine |
| 226 | (3-Aminopropyl)triethoxysilan | Cobalamine |
| 227 | -(2-Aminoethyl)trimethoxysilane | Cobalamine |
| 228 | -(2-Aminoethyl)triethoxysilane | Cobalamine |
| 229 | -(3-Dimethylaminopropyl)trimethoxysilane | Cobalamine |
| 230 | (3-Dimethylaminopropyl)triethoxysilane | Cobalamine |
| 231 | (2-Dimethylaminoethyl)trimethoxysilane | Cobalamine |
| 232 | (2-Dimethylaminoethyl)triethoxysilane | Cobalamine |
| 233 | 3-(trimethoxysilyl)-N-[3-(trimethoxysilyl)propyl]-1-propanamine | Cobalamine |
| 234 | 3-(Triethoxysilyl)-N-[3-(triethoxysilyl)propyl]-1-propanamine | Cobalamine |
| 235 | N-methyl-3-(trimethoxysilyl)-N-[3-(trimethoxysilyl)propyl]-1-propanamine | Cobalamine |
| 236 | N-Methyl-3-(triethoxysilyl)-N-[3-(triethoxysilyl)propyl]-1-propanamine | Cobalamine |
| 237 | 2-[Bis[3-(trimethoxysilyl)propyl]amino]-ethanol | Cobalamine |
| 238 | 2-[bis[3-(triethoxysilyl)propyl]amino]ethanol | Cobalamine |
| 239 | 3-(Trimethoxysilyl)-N,N-bis[3-(trimethoxysilyl)propyl]-1-propanamine | Cobalamine |
| 240 | 3-(Triethoxysilyl)-N,N-bis[3-(triethoxysilyl)propyl]-1-propanamine | Cobalamine |
| 241 | N1,N1-Bis[3-(trimethoxysilyl)propyl]-1,2-ethanediamine | Cobalamine |
| 242 | N1,N1-Bis[3-(triethoxysilyl)propyl]-1,2-ethanediamine | Cobalamine |
| 243 | N,N-Bis[3-(trimethoxysilyl)propyl]-2-propen-1-amine | Cobalamine |
| 244 | N,N-Bis[3-(triethoxysilyl)propyl]-2-propen-1-amine | Cobalamine |
| 245 | Methyltrimethoxysilane | Cobalamine |
| 246 | Methyltriethoxysilane | Cobalamine |
| 247 | Ethyltrimethoxysilane | Cobalamine |
| 248 | Ethyltriethoxysilane | Cobalamine |
| 249 | Octyltrimethoxysilane | Cobalamine |
| 250 | Octyltriethoxysilane | Cobalamine |
| 251 | Dodecyltrimethoxysilane | Cobalamine |
| 252 | Dodecyltriethoxysilane | Cobalamine |
| 253 | -(3-Aminopropyl)trimethoxysilane | Ascorbic acid |
| 254 | (3-Aminopropyl)triethoxysilan | Ascorbic acid |
| 255 | -(2-Aminoethyl)trimethoxysilane | Ascorbic acid |
| 256 | -(2-Aminoethyl)triethoxysilane | Ascorbic acid |
| 257 | -(3-Dimethylaminopropyl)trimethoxysilane | Ascorbic acid |
| 258 | (3-Dimethylaminopropyl)triethoxysilane | Ascorbic acid |
| 259 | (2-Dimethylaminoethyl)trimethoxysilane | Ascorbic acid |
| 260 | (2-Dimethylaminoethyl)triethoxysilane | Ascorbic acid |
| 261 | 3-(trimethoxysilyl)-N-[3-(trimethoxysilyl)propyl]-1-propanamine | Ascorbic acid |
| 262 | 3-(Triethoxysilyl)-N-[3-(triethoxysilyl)propyl]-1-propanamine | Ascorbic acid |
| 263 | N-methyl-3-(trimethoxysilyl)-N-[3-(trimethoxysilyl)propyl]-1-propanamine | Ascorbic acid |
| 264 | N-Methyl-3-(triethoxysilyl)-N-[3-(triethoxysilyl)propyl]-1-propanamine | Ascorbic acid |
| 265 | 2-[Bis[3-(trimethoxysilyl)propyl]amino]-ethanol | Ascorbic acid |
| 266 | 2-[bis[3-(triethoxysilyl)propyl]amino]ethanol | Ascorbic acid |
| 267 | 3-(Trimethoxysilyl)-N,N-bis[3-(trimethoxysilyl)propyl]-1-propanamine | Ascorbic acid |
| 268 | 3-(Triethoxysilyl)-N,N-bis[3-(triethoxysilyl)propyl]-1-propanamine | Ascorbic acid |
| 269 | N1,N1-Bis[3-(trimethoxysilyl)propyl]-1,2-ethanediamine | Ascorbic acid |
| 270 | N1,N1-Bis[3-(triethoxysilyl)propyl]-1,2-ethanediamine | Ascorbic acid |
| 271 | N,N-Bis[3-(trimethoxysilyl)propyl]-2-propen-1-amine | Ascorbic acid |
| 272 | N,N-Bis[3-(triethoxysilyl)propyl]-2-propen-1-amine | Ascorbic acid |
| 273 | Methyltrimethoxysilane | Ascorbic acid |
| 274 | Methyltriethoxysilane | Ascorbic acid |
| 275 | Ethyltrimethoxysilane | Ascorbic acid |
| 276 | Ethyltriethoxysilane | Ascorbic acid |
| 277 | Octyltrimethoxysilane | Ascorbic acid |
| 278 | Octyltriethoxysilane | Ascorbic acid |
| 279 | Dodecyltrimethoxysilane | Ascorbic acid |
| 280 | Dodecyltriethoxysilane | Ascorbic acid |
| 281 | -(3-Aminopropyl)trimethoxysilane | Cholecalciferol |
| 282 | (3-Aminopropyl)triethoxysilan | Cholecalciferol |
| 283 | -(2-Aminoethyl)trimethoxysilane | Cholecalciferol |
| 284 | -(2-Aminoethyl)triethoxysilane | Cholecalciferol |
| 285 | -(3-Dimethylaminopropyl)trimethoxysilane | Cholecalciferol |
| 286 | (3-Dimethylaminopropyl)triethoxysilane | Cholecalciferol |
| 287 | (2-Dimethylaminoethyl)trimethoxysilane | Cholecalciferol |
| 288 | (2-Dimethylaminoethyl)triethoxysilane | Cholecalciferol |
| 289 | 3-(trimethoxysilyl)-N-[3-(trimethoxysilyl)propyl]-1-propanamine | Cholecalciferol |
| 290 | 3-(Triethoxysilyl)-N-[3-(triethoxysilyl)propyl]-1-propanamine | Cholecalciferol |
| 291 | N-methyl-3-(trimethoxysilyl)-N-[3-(trimethoxysilyl)propyl]-1-propanamine | Cholecalciferol |
| 292 | N-Methyl-3-(triethoxysilyl)-N-[3-(triethoxysilyl)propyl]-1-propanamine | Cholecalciferol |
| 293 | 2-[Bis[3-(trimethoxysilyl)propyl]amino]-ethanol | Cholecalciferol |
| 294 | 2-[bis[3-(triethoxysilyl)propyl]amino]ethanol | Cholecalciferol |
| 295 | 3-(Trimethoxysilyl)-N,N-bis[3-(trimethoxysilyl)propyl]-1-propanamine | Cholecalciferol |
| 296 | 3-(Triethoxysilyl)-N,N-bis[3-(triethoxysilyl)propyl]-1-propanamine | Cholecalciferol |
| 297 | N1,N1-Bis[3-(trimethoxysilyl)propyl]-1,2-ethanediamine | Cholecalciferol |
| 298 | N1,N1-Bis[3-(triethoxysilyl)propyl]-1,2-ethanediamine | Cholecalciferol |
| 299 | N,N-Bis[3-(trimethoxysilyl)propyl]-2-propen-1-amine | Cholecalciferol |
| 300 | N,N-Bis[3-(triethoxysilyl)propyl]-2-propen-1-amine | Cholecalciferol |
| 301 | Methyltrimethoxysilane | Cholecalciferol |
| 302 | Methyltriethoxysilane | Cholecalciferol |
| 303 | Ethyltrimethoxysilane | Cholecalciferol |
| 304 | Ethyltriethoxysilane | Cholecalciferol |
| 305 | Octyltrimethoxysilane | Cholecalciferol |
| 306 | Octyltriethoxysilane | Cholecalciferol |
| 307 | Dodecyltrimethoxysilane | Cholecalciferol |
| 308 | Dodecyltriethoxysilane | Cholecalciferol |
| 309 | -(3-Aminopropyl)trimethoxysilane | Tocopherol |
| 310 | (3-Aminopropyl)triethoxysilan | Tocopherol |
| 311 | -(2-Aminoethyl)trimethoxysilane | Tocopherol |
| 312 | -(2-Aminoethyl)triethoxysilane | Tocopherol |
| 313 | -(3-Dimethylaminopropyl)trimethoxysilane | Tocopherol |
| 314 | (3-Dimethylaminopropyl)triethoxysilane | Tocopherol |
| 315 | (2-Dimethylaminoethyl)trimethoxysilane | Tocopherol |
| 316 | (2-Dimethylaminoethyl)triethoxysilane | Tocopherol |
| 317 | 3-(trimethoxysilyl)-N-[3-(trimethoxysilyl)propyl]-1-propanamine | Tocopherol |
| 318 | 3-(Triethoxysilyl)-N-[3-(triethoxysilyl)propyl]-1-propanamine | Tocopherol |
| 319 | N-methyl-3-(trimethoxysilyl)-N-[3-(trimethoxysilyl)propyl]-1-propanamine | Tocopherol |
| 320 | N-Methyl-3-(triethoxysilyl)-N-[3-(triethoxysilyl)propyl]-1-propanamine | Tocopherol |
| 321 | 2-[Bis[3-(trimethoxysilyl)propyl]amino]-ethanol | Tocopherol |
| 322 | 2-[bis[3-(triethoxysilyl)propyl]amino]ethanol | Tocopherol |
| 323 | 3-(Trimethoxysilyl)-N,N-bis[3-(trimethoxysilyl)propyl]-1-propanamine | Tocopherol |
| 324 | 3-(Triethoxysilyl)-N,N-bis[3-(triethoxysilyl)propyl]-1-propanamine | Tocopherol |
| 325 | N1,N1-Bis[3-(trimethoxysilyl)propyl]-1,2-ethanediamine | Tocopherol |
| 326 | N1,N1-Bis[3-(triethoxysilyl)propyl]-1,2-ethanediamine | Tocopherol |
| 327 | N,N-Bis[3-(trimethoxysilyl)propyl]-2-propen-1-amine | Tocopherol |
| 328 | N,N-Bis[3-(triethoxysilyl)propyl]-2-propen-1-amine | Tocopherol |
| 329 | Methyltrimethoxysilane | Tocopherol |
| 330 | Methyltriethoxysilane | Tocopherol |
| 331 | Ethyltrimethoxysilane | Tocopherol |
| 332 | Ethyltriethoxysilane | Tocopherol |
| 333 | Octyltrimethoxysilane | Tocopherol |
| 334 | Octyltriethoxysilane | Tocopherol |
| 335 | Dodecyltrimethoxysilane | Tocopherol |
| 336 | Dodecyltriethoxysilane | Tocopherol |
| 337 | Propyltrimethoxysilane | Retinol |
| 338 | Propyltriethoxysilane | Retinol |
| 339 | Hexyltrimethoxysilane | Retinol |
| 340 | Hexyltriethoxysilane | Retinol |
| 341 | Octadecyltriethoxysilane | Retinol |
| 342 | Octadecyltriethoxysilane | Retinol |
| 343 | (3-Aminopropyl)triethoxysilane + methyltrimethoxysilane | Retinol |
| 344 | (3-Aminopropyl)triethoxysilane + methyltriethoxysilane | Retinol |
| 345 | (3-Aminopropyl)triethoxysilane + ethyltrimethoxysilane | Retinol |
| 346 | (3-Aminopropyl)triethoxysilane + ethyltriethoxysilane | Retinol |
| 347 | (3-Aminopropyl)triethoxysilane + propyltrimethoxysilane | Retinol |
| 348 | (3-Aminopropyl)triethoxysilane + propyltriethoxysilane | Retinol |
| 349 | (3-Aminopropyl)triethoxysilane + hexyltrimethoxysilane | Retinol |
| 350 | (3-aminopropyl)triethoxysilane + hexyltriethoxysilane | Retinol |
| 351 | (3-Aminopropyl)triethoxysilane + octyltrimethoxysilane | Retinol |
| 352 | (3-Aminopropyl)triethoxysilane + Octyltriethoxysilane | Retinol |
| 353 | (3-Aminopropyl)triethoxysilane + dodecyltrimethoxysilane | Retinol |
| 354 | (3-Aminopropyl)triethoxysilane + dodecyltriethoxysilane | Retinol |
| 355 | (3-aminopropyl)triethoxysilane + octadecyltrimethoxysilane | Retinol |
| 356 | (3-Aminopropyl)triethoxysilane + Octadecyltriethoxysilane | Retinol |
| 357 | Propyltrimethoxysilane | Thiamine |
| 358 | Propyltriethoxysilane | Thiamine |
| 359 | Hexyltrimethoxysilane | Thiamine |
| 360 | Hexyltriethoxysilane | Thiamine |
| 361 | Octadecyltriethoxysilane | Thiamine |
| 362 | Octadecyltriethoxysilane | Thiamine |
| 363 | (3-Aminopropyl)triethoxysilane + methyltrimethoxysilane | Thiamine |
| 364 | (3-Aminopropyl)triethoxysilane + methyltriethoxysilane | Thiamine |
| 365 | (3-Aminopropyl)triethoxysilane + ethyltrimethoxysilane | Thiamine |
| 366 | (3-Aminopropyl)triethoxysilane + ethyltriethoxysilane | Thiamine |
| 367 | (3-Aminopropyl)triethoxysilane + propyltrimethoxysilane | Thiamine |
| 368 | (3-Aminopropyl)triethoxysilane + propyltriethoxysilane | Thiamine |
| 369 | (3-Aminopropyl)triethoxysilane + hexyltrimethoxysilane | Thiamine |
| 370 | (3-aminopropyl)triethoxysilane + hexyltriethoxysilane | Thiamine |
| 371 | (3-Aminopropyl)triethoxysilane + octyltrimethoxysilane | Thiamine |
| 372 | (3-Aminopropyl)triethoxysilane + Octyltriethoxysilane | Thiamine |
| 373 | (3-Aminopropyl)triethoxysilane + dodecyltrimethoxysilane | Thiamine |
| 374 | (3-Aminopropyl)triethoxysilane + dodecyltriethoxysilane | Thiamine |
| 375 | (3-aminopropyl)triethoxysilane + octadecyltrimethoxysilane | Thiamine |
| 376 | (3-Aminopropyl)triethoxysilane + Octadecyltriethoxysilane | Thiamine |
| 377 | Propyltrimethoxysilane | Riboflavin |
| 378 | Propyltriethoxysilane | Riboflavin |
| 379 | Hexyltrimethoxysilane | Riboflavin |
| 380 | Hexyltriethoxysilane | Riboflavin |
| 381 | Octadecyltriethoxysilane | Riboflavin |
| 382 | Octadecyltriethoxysilane | Riboflavin |
| 383 | (3-Aminopropyl)triethoxysilane + methyltrimethoxysilane | Riboflavin |
| 384 | (3-Aminopropyl)triethoxysilane + methyltriethoxysilane | Riboflavin |
| 385 | (3-Aminopropyl)triethoxysilane + ethyltrimethoxysilane | Riboflavin |
| 386 | (3-Aminopropyl)triethoxysilane + ethyltriethoxysilane | Riboflavin |
| 387 | (3-Aminopropyl)triethoxysilane + propyltrimethoxysilane | Riboflavin |
| 388 | (3-Aminopropyl)triethoxysilane + propyltriethoxysilane | Riboflavin |
| 389 | (3-Aminopropyl)triethoxysilane + hexyltrimethoxysilane | Riboflavin |
| 390 | (3-aminopropyl)triethoxysilane + hexyltriethoxysilane | Riboflavin |
| 391 | (3-Aminopropyl)triethoxysilane + octyltrimethoxysilane | Riboflavin |
| 392 | (3-Aminopropyl)triethoxysilane + Octyltriethoxysilane | Riboflavin |
| 393 | (3-Aminopropyl)triethoxysilane + dodecyltrimethoxysilane | Riboflavin |
| 394 | (3-Aminopropyl)triethoxysilane + dodecyltriethoxysilane | Riboflavin |
| 395 | (3-aminopropyl)triethoxysilane + octadecyltrimethoxysilane | Riboflavin |
| 396 | (3-Aminopropyl)triethoxysilane + Octadecyltriethoxysilane | Riboflavin |
| 397 | Propyltrimethoxysilane | Niacin |
| 398 | Propyltriethoxysilane | Niacin |
| 399 | Hexyltrimethoxysilane | Niacin |
| 400 | Hexyltriethoxysilane | Niacin |
| 401 | Octadecyltriethoxysilane | Niacin |
| 402 | Octadecyltriethoxysilane | Niacin |
| 403 | (3-Aminopropyl)triethoxysilane + methyltrimethoxysilane | Niacin |
| 404 | (3-Aminopropyl)triethoxysilane + methyltriethoxysilane | Niacin |
| 405 | (3-Aminopropyl)triethoxysilane + ethyltrimethoxysilane | Niacin |
| 406 | (3-Aminopropyl)triethoxysilane + ethyltriethoxysilane | Niacin |
| 407 | (3-Aminopropyl)triethoxysilane + propyltrimethoxysilane | Niacin |
| 408 | (3-Aminopropyl)triethoxysilane + propyltriethoxysilane | Niacin |
| 409 | (3-Aminopropyl)triethoxysilane + hexyltrimethoxysilane | Niacin |
| 410 | (3-aminopropyl)triethoxysilane + hexyltriethoxysilane | Niacin |
| 411 | (3-Aminopropyl)triethoxysilane + octyltrimethoxysilane | Niacin |
| 412 | (3-Aminopropyl)triethoxysilane + Octyltriethoxysilane | Niacin |
| 413 | (3-Aminopropyl)triethoxysilane + dodecyltrimethoxysilane | Niacin |
| 414 | (3-Aminopropyl)triethoxysilane + dodecyltriethoxysilane | Niacin |
| 415 | (3-aminopropyl)triethoxysilane + octadecyltrimethoxysilane | Niacin |
| 416 | (3-Aminopropyl)triethoxysilane + Octadecyltriethoxysilane | Niacin |
| 417 | Propyltrimethoxysilane | Pantothenic acid |
| 418 | Propyltriethoxysilane | Pantothenic acid |
| 419 | Hexyltrimethoxysilane | Pantothenic acid |
| 420 | Hexyltriethoxysilane | Pantothenic acid |
| 421 | Octadecyltriethoxysilane | Pantothenic acid |
| 422 | Octadecyltriethoxysilane | Pantothenic acid |
| 423 | (3-Aminopropyl)triethoxysilane + methyltrimethoxysilane | Pantothenic acid |
| 424 | (3-Aminopropyl)triethoxysilane + methyltriethoxysilane | Pantothenic acid |
| 425 | (3-Aminopropyl)triethoxysilane + ethyltrimethoxysilane | Pantothenic acid |
| 426 | (3-Aminopropyl)triethoxysilane + ethyltriethoxysilane | Pantothenic acid |
| 427 | (3-Aminopropyl)triethoxysilane + propyltrimethoxysilane | Pantothenic acid |
| 428 | (3-Aminopropyl)triethoxysilane + propyltriethoxysilane | Pantothenic acid |
| 429 | (3-Aminopropyl)triethoxysilane + hexyltrimethoxysilane | Pantothenic acid |
| 430 | (3-aminopropyl)triethoxysilane + hexyltriethoxysilane | Pantothenic acid |
| 431 | (3-Aminopropyl)triethoxysilane + octyltrimethoxysilane | Pantothenic acid |
| 432 | (3-Aminopropyl)triethoxysilane + Octyltriethoxysilane | Pantothenic acid |
| 433 | (3-Aminopropyl)triethoxysilane + dodecyltrimethoxysilane | Pantothenic acid |
| 434 | (3-Aminopropyl)triethoxysilane + dodecyltriethoxysilane | Pantothenic acid |
| 435 | (3-aminopropyl)triethoxysilane + octadecyltrimethoxysilane | Pantothenic acid |
| 436 | (3-Aminopropyl)triethoxysilane + Octadecyltriethoxysilane | Pantothenic acid |
| 437 | Propyltrimethoxysilane | Pyridoxine |
| 438 | Propyltriethoxysilane | Pyridoxine |
| 439 | Hexyltrimethoxysilane | Pyridoxine |
| 440 | Hexyltriethoxysilane | Pyridoxine |
| 441 | Octadecyltriethoxysilane | Pyridoxine |
| 442 | Octadecyltriethoxysilane | Pyridoxine |
| 443 | (3-Aminopropyl)triethoxysilane + methyltrimethoxysilane | Pyridoxine |
| 444 | (3-Aminopropyl)triethoxysilane + methyltriethoxysilane | Pyridoxine |
| 445 | (3-Aminopropyl)triethoxysilane + ethyltrimethoxysilane | Pyridoxine |
| 446 | (3-Aminopropyl)triethoxysilane + ethyltriethoxysilane | Pyridoxine |
| 447 | (3-Aminopropyl)triethoxysilane + propyltrimethoxysilane | Pyridoxine |
| 448 | (3-Aminopropyl)triethoxysilane + propyltriethoxysilane | Pyridoxine |
| 449 | (3-Aminopropyl)triethoxysilane + hexyltrimethoxysilane | Pyridoxine |
| 450 | (3-aminopropyl)triethoxysilane + hexyltriethoxysilane | Pyridoxine |
| 451 | (3-Aminopropyl)triethoxysilane + octyltrimethoxysilane | Pyridoxine |
| 452 | (3-Aminopropyl)triethoxysilane + Octyltriethoxysilane | Pyridoxine |
| 453 | (3-Aminopropyl)triethoxysilane + dodecyltrimethoxysilane | Pyridoxine |
| 454 | (3-Aminopropyl)triethoxysilane + dodecyltriethoxysilane | Pyridoxine |
| 455 | (3-aminopropyl)triethoxysilane + octadecyltrimethoxysilane | Pyridoxine |
| 456 | (3-Aminopropyl)triethoxysilane + Octadecyltriethoxysilane | Pyridoxine |
| 457 | Propyltrimethoxysilane | Biotin |
| 458 | Propyltriethoxysilane | Biotin |
| 459 | Hexyltrimethoxysilane | Biotin |
| 460 | Hexyltriethoxysilane | Biotin |
| 461 | Octadecyltriethoxysilane | Biotin |
| 462 | Octadecyltriethoxysilane | Biotin |
| 463 | (3-Aminopropyl)triethoxysilane + methyltrimethoxysilane | Biotin |
| 464 | (3-Aminopropyl)triethoxysilane + methyltriethoxysilane | Biotin |
| 465 | (3-Aminopropyl)triethoxysilane + ethyltrimethoxysilane | Biotin |
| 466 | (3-Aminopropyl)triethoxysilane + ethyltriethoxysilane | Biotin |
| 467 | (3-Aminopropyl)triethoxysilane + propyltrimethoxysilane | Biotin |
| 468 | (3-Aminopropyl)triethoxysilane + propyltriethoxysilane | Biotin |
| 469 | (3-Aminopropyl)triethoxysilane + hexyltrimethoxysilane | Biotin |
| 470 | (3-aminopropyl)triethoxysilane + hexyltriethoxysilane | Biotin |
| 471 | (3-Aminopropyl)triethoxysilane + octyltrimethoxysilane | Biotin |
| 472 | (3-Aminopropyl)triethoxysilane + Octyltriethoxysilane | Biotin |
| 473 | (3-Aminopropyl)triethoxysilane + dodecyltrimethoxysilane | Biotin |
| 474 | (3-Aminopropyl)triethoxysilane + dodecyltriethoxysilane | Biotin |
| 475 | (3-aminopropyl)triethoxysilane + octadecyltrimethoxysilane | Biotin |
| 476 | (3-Aminopropyl)triethoxysilane + Octadecyltriethoxysilane | Biotin |
| 477 | Propyltrimethoxysilane | Folic acid |
| 478 | Propyltriethoxysilane | Folic acid |
| 479 | Hexyltrimethoxysilane | Folic acid |
| 480 | Hexyltriethoxysilane | Folic acid |
| 481 | Octadecyltriethoxysilane | Folic acid |
| 482 | Octadecyltriethoxysilane | Folic acid |
| 483 | (3-Aminopropyl)triethoxysilane + methyltrimethoxysilane | Folic acid |
| 484 | (3-Aminopropyl)triethoxysilane + methyltriethoxysilane | Folic acid |
| 485 | (3-Aminopropyl)triethoxysilane + ethyltrimethoxysilane | Folic acid |
| 486 | (3-Aminopropyl)triethoxysilane + ethyltriethoxysilane | Folic acid |
| 487 | (3-Aminopropyl)triethoxysilane + propyltrimethoxysilane | Folic acid |
| 488 | (3-Aminopropyl)triethoxysilane + propyltriethoxysilane | Folic acid |
| 489 | (3-Aminopropyl)triethoxysilane + hexyltrimethoxysilane | Folic acid |
| 490 | (3-aminopropyl)triethoxysilane + hexyltriethoxysilane | Folic acid |
| 491 | (3-Aminopropyl)triethoxysilane + octyltrimethoxysilane | Folic acid |
| 492 | (3-Aminopropyl)triethoxysilane + Octyltriethoxysilane | Folic acid |
| 493 | (3-Aminopropyl)triethoxysilane + dodecyltrimethoxysilane | Folic acid |
| 494 | (3-Aminopropyl)triethoxysilane + dodecyltriethoxysilane | Folic acid |
| 495 | (3-aminopropyl)triethoxysilane + octadecyltrimethoxysilane | Folic acid |
| 496 | (3-Aminopropyl)triethoxysilane + Octadecyltriethoxysilane | Folic acid |
| 497 | Propyltrimethoxysilane | Cobalamine |
| 498 | Propyltriethoxysilane | Cobalamine |
| 499 | Hexyltrimethoxysilane | Cobalamine |
| 500 | Hexyltriethoxysilane | Cobalamine |
| 501 | Octadecyltriethoxysilane | Cobalamine |
| 502 | Octadecyltriethoxysilane | Cobalamine |
| 503 | (3-Aminopropyl)triethoxysilane + methyltrimethoxysilane | Cobalamine |
| 504 | (3-Aminopropyl)triethoxysilane + methyltriethoxysilane | Cobalamine |
| 505 | (3-Aminopropyl)triethoxysilane + ethyltrimethoxysilane | Cobalamine |
| 506 | (3-Aminopropyl)triethoxysilane + ethyltriethoxysilane | Cobalamine |
| 507 | (3-Aminopropyl)triethoxysilane + propyltrimethoxysilane | Cobalamine |
| 508 | (3-Aminopropyl)triethoxysilane + propyltriethoxysilane | Cobalamine |
| 509 | (3-Aminopropyl)triethoxysilane + hexyltrimethoxysilane | Cobalamine |
| 510 | (3-aminopropyl)triethoxysilane + hexyltriethoxysilane | Cobalamine |
| 511 | (3-Aminopropyl)triethoxysilane + octyltrimethoxysilane | Cobalamine |
| 512 | (3-Aminopropyl)triethoxysilane + Octyltriethoxysilane | Cobalamine |
| 513 | (3-Aminopropyl)triethoxysilane + dodecyltrimethoxysilane | Cobalamine |
| 514 | (3-Aminopropyl)triethoxysilane + dodecyltriethoxysilane | Cobalamine |
| 515 | (3-aminopropyl)triethoxysilane + octadecyltrimethoxysilane | Cobalamine |
| 516 | (3-Aminopropyl)triethoxysilane + Octadecyltriethoxysilane | Cobalamine |
| 517 | Propyltrimethoxysilane | Ascorbic acid |
| 518 | Propyltriethoxysilane | Ascorbic acid |
| 519 | Hexyltrimethoxysilane | Ascorbic acid |
| 520 | Hexyltriethoxysilane | Ascorbic acid |
| 521 | Octadecyltriethoxysilane | Ascorbic acid |
| 522 | Octadecyltriethoxysilane | Ascorbic acid |
| 523 | (3-Aminopropyl)triethoxysilane + methyltrimethoxysilane | Ascorbic acid |
| 524 | (3-Aminopropyl)triethoxysilane + methyltriethoxysilane | Ascorbic acid |
| 525 | (3-Aminopropyl)triethoxysilane + ethyltrimethoxysilane | Ascorbic acid |
| 526 | (3-Aminopropyl)triethoxysilane + ethyltriethoxysilane | Ascorbic acid |
| 527 | (3-Aminopropyl)triethoxysilane + propyltrimethoxysilane | Ascorbic acid |
| 528 | (3-Aminopropyl)triethoxysilane + propyltriethoxysilane | Ascorbic acid |
| 529 | (3-Aminopropyl)triethoxysilane + hexyltrimethoxysilane | Ascorbic acid |
| 530 | (3-aminopropyl)triethoxysilane + hexyltriethoxysilane | Ascorbic acid |
| 531 | (3-Aminopropyl)triethoxysilane + octyltrimethoxysilane | Ascorbic acid |
| 532 | (3-Aminopropyl)triethoxysilane + Octyltriethoxysilane | Ascorbic acid |
| 533 | (3-Aminopropyl)triethoxysilane + dodecyltrimethoxysilane | Ascorbic acid |
| 534 | (3-Aminopropyl)triethoxysilane + dodecyltriethoxysilane | Ascorbic acid |
| 535 | (3-aminopropyl)triethoxysilane + octadecyltrimethoxysilane | Ascorbic acid |
| 536 | (3-Aminopropyl)triethoxysilane + Octadecyltriethoxysilane | Ascorbic acid |
| 537 | Propyltrimethoxysilane | Cholecalciferol |
| 538 | Propyltriethoxysilane | Cholecalciferol |
| 539 | Hexyltrimethoxysilane | Cholecalciferol |
| 540 | Hexyltriethoxysilane | Cholecalciferol |
| 541 | Octadecyltriethoxysilane | Cholecalciferol |
| 542 | Octadecyltriethoxysilane | Cholecalciferol |
| 543 | (3-Aminopropyl)triethoxysilane + methyltrimethoxysilane | Cholecalciferol |
| 544 | (3-Aminopropyl)triethoxysilane + methyltriethoxysilane | Cholecalciferol |
| 545 | (3-Aminopropyl)triethoxysilane + ethyltrimethoxysilane | Cholecalciferol |
| 546 | (3-Aminopropyl)triethoxysilane + ethyltriethoxysilane | Cholecalciferol |
| 547 | (3-Aminopropyl)triethoxysilane + propyltrimethoxysilane | Cholecalciferol |
| 548 | (3-Aminopropyl)triethoxysilane + propyltriethoxysilane | Cholecalciferol |
| 549 | (3-Aminopropyl)triethoxysilane + hexyltrimethoxysilane | Cholecalciferol |
| 550 | (3-aminopropyl)triethoxysilane + hexyltriethoxysilane | Cholecalciferol |
| 551 | (3-Aminopropyl)triethoxysilane + octyltrimethoxysilane | Cholecalciferol |
| 552 | (3-Aminopropyl)triethoxysilane + Octyltriethoxysilane | Cholecalciferol |
| 553 | (3-Aminopropyl)triethoxysilane + dodecyltrimethoxysilane | Cholecalciferol |
| 554 | (3-Aminopropyl)triethoxysilane + dodecyltriethoxysilane | Cholecalciferol |
| 555 | (3-aminopropyl)triethoxysilane + octadecyltrimethoxysilane | Cholecalciferol |
| 556 | (3-Aminopropyl)triethoxysilane + Octadecyltriethoxysilane | Cholecalciferol |
| 557 | Propyltrimethoxysilane | Tocopherol |
| 558 | Propyltriethoxysilane | Tocopherol |
| 559 | Hexyltrimethoxysilane | Tocopherol |
| 560 | Hexyltriethoxysilane | Tocopherol |
| 561 | Octadecyltriethoxysilane | Tocopherol |
| 562 | Octadecyltriethoxysilane | Tocopherol |
| 563 | (3-Aminopropyl)triethoxysilane + methyltrimethoxysilane | Tocopherol |
| 564 | (3-Aminopropyl)triethoxysilane + methyltriethoxysilane | Tocopherol |
| 565 | (3-Aminopropyl)triethoxysilane + ethyltrimethoxysilane | Tocopherol |
| 566 | (3-Aminopropyl)triethoxysilane + ethyltriethoxysilane | Tocopherol |
| 567 | (3-Aminopropyl)triethoxysilane + propyltrimethoxysilane | Tocopherol |
| 568 | (3-Aminopropyl)triethoxysilane + propyltriethoxysilane | Tocopherol |
| 569 | (3-Aminopropyl)triethoxysilane + hexyltrimethoxysilane | Tocopherol |
| 570 | (3-aminopropyl)triethoxysilane + hexyltriethoxysilane | Tocopherol |
| 571 | (3-Aminopropyl)triethoxysilane + octyltrimethoxysilane | Tocopherol |
| 572 | (3-Aminopropyl)triethoxysilane + Octyltriethoxysilane | Tocopherol |
| 573 | (3-Aminopropyl)triethoxysilane + dodecyltrimethoxysilane | Tocopherol |
| 574 | (3-Aminopropyl)triethoxysilane + dodecyltriethoxysilane | Tocopherol |
| 575 | (3-aminopropyl)triethoxysilane + octadecyltrimethoxysilane | Tocopherol |
| 576 | (3-Aminopropyl)triethoxysilane + Octadecyltriethoxysilane | Tocopherol |

The combinations in the above table represent active ingredient combinations that are combined in cosmetic products with other components described above.

The active ingredient combination of at least one organic silicon compound and the antioxidant may already be present in the agent for treating a keratinous material. In this embodiment, the agent for treating a keratinous material is already distributed in a form ready for use. To provide a formulation that is as stable as possible during storage, the agent itself is in an exemplary embodiment packaged with low or no water.

Alternatively, the at least one organic silicon compound is added a maximum of 12 hours, in an exemplary embodiment a maximum of 6 hours, such as a maximum of 3 hours, even such as a maximum of 1 hour prior to application of the keratinous material treatment composition to a base comprising all the ingredients of the keratinous material treatment composition except the at least one organic silicon compound.

Furthermore, alternatively, the organic silicon compound and another component b) are added to a cosmetic product only shortly before use, i.e., 1 minute to 12 hours, in an exemplary embodiment from about 2 minutes to about 6 hours, for example from about 1 minute to about 3 hours, in particular embodiments from about 1 minute to about 1 hour.

In a further alternative, the AMEO or the bis(triethoxysilylpropyl)amine is added to an aqueous solution which is applied to the hair and, in a second step, an aqueous solution or a cosmetic composition containing the further component b) is applied to the hair.

For example, the user may first mix or shake an agent (u) comprising the organic silicon compound(s) with an agent (β) comprising the remaining ingredients of the agent for treating a keratinous material. The user can now apply this mixture of (α) and (β)—either directly after its preparation or after a short reaction time of 1 minute to 20 minutes—to the keratinous materials. The agent (β) may contain water, in particular water in an amount >30% by weight, based on the total weight of the agent for treating keratinous materials.

Another object of the present application is the use of a cosmetic composition as contemplated herein for treating a keratinous material for the care of keratinous material, for reducing and/or preventing harmful effects of air and water contaminants on keratinous material, for reducing and/or preventing the formation of free radicals by air and water contaminants on a keratinous material, and/or for rendering harmless free radicals formed by air and water contaminants on a keratinous material.

About further suitable embodiments of use, the same applies mutatis mutandis as to the cosmetic agents.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for treating a keratinous material comprising:
a) at least one organic silicon compound; and
b) at least one antioxidant.

2. The cosmetic composition for treating a keratinous material according to claim 1,
wherein:
the at least one organic silicon compound comprises a compound of the formula (I) and/or (II),
wherein in the organic silicon compound of formula (I)
R₁R₂N-L-Si(OR₃)ₐ(R₄)_{b} (I),
R₁, R₂ both represent a hydrogen atom,
L represents a linear, two-band C₁-C₆-alkylene group,
R₃, R₄ independently represent a methyl group or an ethyl group,
a stands for the number 3 and
b stands for the number 0, and
wherein in the organic silicon compound of formula (II)
(R₅₀)_{c}(R₆)_{d}Si-(A)ₑ[NR₇(A′)]_{f}-[O-(A″)]_{g}-[NR₈-(A′″)]ₕ-Si(R₆′)_{d′}(OR₅′)_{c′} (II),
R₅, R_{5′}, R_{5″}, R₆, R_{6′} and R_{6″} independently represent a C₁-C₆ alkyl group,
A, A′, A″, A′″ and A″″ independently represent a linear or branched C₁-C₂₀ divalent alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
-(A″″)-Si(R₆″)_{d″}(OR₅″)_{c″} (III),
c stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
with the proviso that at least one of the residues from e, f, g, and h is different from 0.

3. The cosmetic composition for treating a keratinous material according to claim 1,
wherein
the agent for treating a keratinous material comprises at least one organic silicon compound of formula (I) selected from the group of
(3-Aminopropyl)trimethoxysilane
(3-Aminopropyl)triethoxysilane
(2-Aminoethyl)trimethoxysilane
(2-Aminoethyl)triethoxysilane
(3-Dimethylaminopropyl)trimethoxysilane
(3-Dimethylaminopropyl)triethoxysilane
(2-dimethylaminoethyl)trimethoxysilane and
(2-dimethylaminoethyl)triethoxysilane,
or wherein
the agent for treating a keratinous material comprises at least one organic silicon compound of formula (II) selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and
N,N-bis[3-(triethoxysilyl)propyl]-2-propen-1-amine.

4. The cosmetic composition for treating a keratinous material according to claim 1, wherein the organic silicon compound of formula (I) is present in the cosmetic composition in an amount of from about 0.01 to about 10% by weight, based on the total weight of the cosmetic composition, and/or wherein the organic silicon compound of formula (II) is present in an amount of from about 0.01 to about 10% by weight, based on the total weight of the cosmetic composition, and/or wherein the organic silicon compound of formula (I) is 3-(triethoxysilyl)-N-[3-(triethoxysilyl)propyl]-1-propanamine and/or the organic silicon compound of formula (II) is (3-aminopropyl)triethoxysilane.

5. The cosmetic composition for treating a keratinous material according to claim 1, wherein the composition for treating a keratinous material comprises at least one organic silicon compound of formula (IV),
R₉Si(OR₁₀)ₖ(R₁₁)ₘ (IV),
which is selected from the group of
Methyltrimethoxysilane
Methyltriethoxysilane
Ethyltrimethoxysilane
Ethyltriethoxysilane
Propyltrimethoxysilane
Propyltriethoxysilane
Hexyltrimethoxysilane
Hexyltriethoxysilane
Octyltrimethoxysilane
Octyltriethoxysilane
Dodecyltrimethoxysilane
Dodecyltriethoxysilane
Octadecyltrimethoxysilane and
Octadecyltriethoxysilane.

6. The cosmetic composition for treating a keratinous material according to claim 1, wherein the antioxidant is a reducing agent having a redox potential of +0.12 V or less or a radical scavenger, or wherein the antioxidant is selected from the group of phenol derivatives, flavonoids, flavonoid derivatives, anthocyanins, preferably pelargonidin, cyanidin, peonidin, paeonidin, rosinidin, delphinidin, petunidin or malvidin, carotenoids, retinol, thiamin, riboflavin, niacin, nicotinamide, nicotinic acid, pantothenic acid, pyridoxine, pyridoxal, pyridoxamine, biotin, folic acid, cobalamin, ascorbic acid, sulfurous acid salts, cholecalciferol, tocopherol, phylloquinone, menaquinone, linolenic acid, amygdalin, butylhydroxyanisole, butylhydroxytoluene and mixtures thereof.

7. The cosmetic composition for treating a keratinous material according to claim 1, wherein the antioxidant is present in the cosmetic composition in an amount of from about 0.1 to about 10% by weight, based on the total weight of the cosmetic composition.

8. The cosmetic composition for treating a keratinous material according to claim 1, wherein the cosmetic composition further comprises a component c) which is a further skin moisturizing agent selected from the group of glycerol, urea, hyaluronic acid, silanol esters of hyaluronic acid, panthenol, taurine, ceramides, phytosterols, aloe vera extracts, creatine, creatinine, sodium hyaluronate, polysaccharides, biosaccharides gum-1, cucumber extracts, butylene glycol, propylene glycol, methyl propanediol, ethylhexyl glycerin, sorbitol, amino acids, amino acid derivatives, lactic acid, lactates, in particular sodium lactate, and/or ethylhexyloxyglycerol.

9. The cosmetic composition for treating a keratinous material according to claim 1, wherein the cosmetic composition for treating a keratinous material comprises at least two organic silicon compounds which are structurally different from one another.

10. The cosmetic composition for treating a keratinous material according to claim 1, wherein the composition for treating a keratinous material comprises—based on the total weight of the composition for treating a keratinous material:
from about 0.5 to about 3 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
from about 3.2 to about 7 wt. % of at least one second organic silicon compound selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilanen, octadecyltrimethoxysilane and octadecyltriethoxysilane.

11. (canceled)
